(19) United States
(12) Patent Application Publication
(10) Pub. No.: US 2026/0083945 A1
(43) Pub. Date: Mar. 26, 2026

(54) VARIABLE ORIFICE FLOW

(71) Applicant: Edwards Lifesciences Corporation, Irvine, CA (US)

(72) Inventors: Hengchu Cao, Irvine, CA (US); Ming H. Wu, Tustin, CA (US); Ralph Schneider, Fallbrook, CA (US); Maximilien Evans Launey, Pasadena, CA (US)

(21) Appl. No.: 19/400,924

(22) Filed: Nov. 25, 2025

Related U.S. Application Data
(63) Continuation of application No. PCT/US2024/031275, filed on May 28, 2024.
(60) Provisional application No. 63/505,418, filed on May 31, 2023.

(51) Int. Cl. A61M 27/00; A61F 2/24; A61F 2/48; A61M 39/24

(52) U.S. Cl. CPC A61M 27/002; A61F 2/2476; A61F 2/482; A61F 2220/0016; A61F 2230/001; A61F 2230/0069; A61F 2250/0007; A61F 2250/001; A61F 2250/0039; A61M 2039/242; A61M 2205/0266; A61M 2205/0272; A61M 2205/04; A61M 2205/3327; A61M 2205/3344; A61M 2205/50; A61M 2210/12; A61M 2230/005; A61M 2230/30

(57) ABSTRACT

Described herein are shunt devices configured to achieve a targeted reduction in peak systolic pressure by percutaneously shunting from the superior vena cava to the right pulmonary artery. The shunt devices include a check valve with a cracking pressure to preserve a minimal transpulmonary pressure. Also described herein are flow diversion devices placed between pulmonary circulation vessels and venous vessels. The disclosed flow diversion devices are physiologically responsive through an adjustable orifice that can be adjusted after initial implantation through a non-invasive or minimally invasive procedure. Disclosed herein are flow control systems that are configured to reduce recruitment of blood from the splanchnic system. The flow control systems include a variable orifice constrictor (e.g., a covered stent) and a controller that delivers energy to the constrictor to reduce the cross-sectional area of the lumen through the variable orifice constrictor.

### CROSS-REFERENCE TO RELATED APPLICATION

This application is a continuation of International Patent Application No. PCT/US2024/031275, filed May 28, 2024, which claims the benefit of U.S. Provisional Application No. 63/505,418, filed May 31, 2023, the complete disclosures of which are hereby incorporated by reference.

### BACKGROUND

### Field

The present disclosure relates to the field of medical devices and procedures.

### Description of the Related Art

Pulmonary hypertension (PH) is a class of ailments involving high blood pressure and it affects the arteries in the lungs and the right side of the heart. PH inflicts at least 2.7 million Americans and complicates common diseases and leads to worsening symptoms and increased mortality. The increased pressure in the blood vessels of the lungs means that the heart has to work harder to pump blood into the lungs. To meet this increased demand, the sympathetic nervous system may recruit more blood from the splanchnic system. Patients with PH are particularly sensitive to these elevated activity levels. A result from the recruitment of blood from the splanchnic system may be pulmonary congestion, which often leads to hospital admission.

### SUMMARY

Described herein are methods and/or devices to adjust blood flow through a vessel, such as the inferior vena cava, or through a shunt pathway, such as between the pulmonary artery and superior vena cava or between the left and right atriums. The disclosed devices form adjustable orifices to regulate blood flow through the orifice.

In some implementations, the disclosed systems are configured to reduce recruitment of blood from the splanchnic system. The disclosed systems include a variable orifice constrictor (e.g., a covered stent) and a controller that delivers energy to the constrictor to reduce the cross-sectional area of the lumen through the variable orifice constrictor. This reduction in cross-section slows down the recruitment of venous blood into the stressed blood volume whereas under normal physiological conditions, exercise or other stimulation would increase the blood volume of the patient which may cause pulmonary congestion.

In some implementations, the techniques described herein relate to a variable orifice flow control system including: an endovascular covered stent device including: two apposition ends; a variable orifice portion connecting the two apposition ends and situated between the apposition ends to form a central lumen; bridge elements coupled to each apposition end; and an actuating element coupled to the bridge elements of each apposition end, wherein the actuating element is configured to change length and the variable orifice portion is configured to change a size of the cross-sectional area of the central lumen due to the change in length of the actuating element; and a control system that is electrically coupled to the actuating element and configured to deliver energy to the actuating element to change the length of the actuating element.

In some implementations, the techniques described herein utilize unique mechanical and shape memory properties related to the martensitic state and the austenitic state of a shape memory alloy. Shape memory alloys, such as Nitinol, exhibit martensitic transformation when cooled below the martensitic transformation start (Mₛ) temperature. Martensitic transformation can also occur by deformation at temperatures at or above Mₛ temperature through stress-induced transformation. An implantable medical device of shape memory alloy exhibits superelastic, self-expanding property when the austenitic reverse transformation finish (A_{f}) temperature is at or below the body temperature. In some implementations, the techniques described here utilize the martensitic state which is induced either thermally by cooling or stress-induced by deformation in the alloy whereby the alloy is at least partially processed such that the starting temperature of the reverse martensitic transformation, e.g., the martensite to R-phase transformation start (R′ₛ) temperature in a two-stage reverse transformation (specific in Nitinol) or the martensite to austenitic transformation start (Aₛ) temperature in a single-stage transformation, is at or above the body temperature. In such implementations, the martensitic portion of the alloy does not exhibit superelastic, self-expanding property. Instead, the alloy in such martensitic state can be plastically deformed to retain the deformed shape. In some implementations, the martensitic state processed as such can be heated to recover the original shape of a shape memory actuator element.

In some implementations, the techniques described herein relate to a variable orifice flow control system, wherein the actuating element is a shape memory alloy. In some implementations, the techniques described herein relate to a variable orifice flow control system, wherein the actuating element is configured to shorten to constrict the central lumen. In some implementations, the techniques described herein relate to a variable orifice control system, wherein the control system is configured to receive physiological signals and to implement a control algorithm that changes the size of the central lumen in response to the physiological signals. In some implementations, the techniques described herein relate to a variable orifice control system, wherein the physiological signals include pressure measurements acquired in a left atrium. In some implementations, the techniques described herein relate to a variable orifice flow control system, wherein the control system is configured to heat the actuating element to constrict the actuating element. In some implementations, the techniques described herein relate to a variable orifice flow control system, wherein the control system is configured to deliver electrical energy to the actuating element to constrict the actuating element. In some implementations, the techniques described herein relate to a variable orifice flow control system further including flexible electrical leads that electrically couple the actuating element to the control system. In some implementations, the techniques described herein relate to a variable orifice flow control system, wherein the actuating element includes a shape memory alloy configured to be in a martensitic state at body temperature. In some implementations, the techniques described herein relate to a variable orifice flow control system, wherein the endovascular covered stent device is configured to be implanted in the inferior vena cava of a patient.

In some implementations, the techniques described herein relate to a shunt device configured to alleviate pressure overload of a heart organ, the shunt device including: an inlet port; an outlet port; two or more leaflets to form the outlet port, the two or more leaflets pre-formed to hold a closed state at or under a threshold pressure differential, the two or more leaflets configured to open to allow blood flow through the shunt device in response to a pressure that exceeds the threshold pressure differential; and two or more commissure posts to couple the two or more leaflets to the shunt device, the two or more commissure posts extending from the inlet port to the outlet port.

In some implementations, the techniques described herein relate to a shunt device, wherein the shunt device includes metallic alloys. In some implementations, the techniques described herein relate to a shunt device, wherein the shunt device includes plastics. In some implementations, the techniques described herein relate to a shunt device, wherein the shunt device is configured for placement at a junction between the superior vena cava and pulmonary artery. In some implementations, the techniques described herein relate to a shunt device, wherein the shunt device is configured to alleviate right ventricle pressure. In some implementations, the techniques described herein relate to a shunt device, wherein the two or more leaflets are configured to remain closed when a pressure differential between the inlet port and the outlet ports is at or below 20 mmHg. In some implementations, the techniques described herein relate to a shunt device, wherein the two or more leaflets are configured to open responsive to the pressure differential exceeding 20 mmHg. In some implementations, the techniques described herein relate to a shunt device further including a structural support frame, the two or more commissure ports extending from the structural support frame, the two or more leaflets coupled to the structural support frame. In some implementations, the techniques described herein relate to a shunt device, wherein the structural support frame includes a flexible portion configured to move with the two or more leaflets. In some implementations, the techniques described herein relate to a shunt device, wherein the two or more leaflets include a biocompatible polymer.

In some implementations, the techniques described herein relate to a shunt device configured to alleviate pressure overload of a heart organ, the shunt device including: an inlet port; an outlet port; a check valve formed from three or more leaflets that form the outlet port, the at least three leaflets pre-formed to hold a closed state at or under a threshold pressure differential, the at least three leaflets configured to open to allow blood flow through the shunt device in response to a pressure that exceeds the threshold pressure differential; at least three commissure posts to couple the at least three leaflets to the shunt device, the at least three commissure posts extending from the inlet port to the outlet port; and at least three cusp posts that regulate opening of the at least three leaflets.

In some implementations, the techniques described herein relate to a shunt device, wherein the shunt device includes metallic alloys. In some implementations, the techniques described herein relate to a shunt device, wherein the shunt device includes plastics. In some implementations, the techniques described herein relate to a shunt device, wherein the shunt device is configured for placement at a junction between the superior vena cava and pulmonary artery, at an interatrial septum of the heart organ, or between a left atrium and coronary sinus of the heart organ. In some implementations, the techniques described herein relate to a shunt device, wherein the check valve includes a variable flow control device that provides a plurality of flow states. In some implementations, the techniques described herein relate to a shunt device, wherein the shunt device is configured to alleviate right ventricle pressure. In some implementations, the techniques described herein relate to a shunt device, wherein the at least three leaflets are configured to remain closed when a pressure differential between the inlet port and the outlet ports is at or below 20 mmHg. In some implementations, the techniques described herein relate to a shunt device, wherein the at least three leaflets are configured to open responsive to the pressure differential exceeding 20 mmHg. In some implementations, the techniques described herein relate to a shunt device further including a structural support frame that forms a tri-leaflet variable orifice support with three stiff posts corresponding to the at least three commissure ports and three flexible posts corresponding to the at least three cusp posts. In some implementations, the techniques described herein relate to a shunt device, wherein the check valve forms a shamrock shape when the check valve is open.

In some implementations, the techniques described herein relate to a shunt device configured to provide a variable orifice, the shunt device including: a shunt frame that forms an inlet port and an outlet port and that is configured to support a shunt opening; a leaflet support structure coupled to the support frame; a leaflet including a soft tissue or polymer membrane, the leaflet coupled to the leaflet support structure, the leaflet support structure configured to deflect in response to a pressure differential or a pressure gradient imposed on the leaflet; and a wrapping membrane configured to wrap the support frame to regulate a flow rate through the shunt device, wherein the leaflet and the leaflet support structure cooperate to form a flapper check valve that provides a variable orifice flow responsive to the pressure gradient imposed on the leaflet.

In some implementations, the techniques described herein relate to a shunt device, wherein the leaflet support structure includes an elastic material or a super-elastic alloy configured so that a cracking pressure of the shunt device can be set to a targeted value. In some implementations, the techniques described herein relate to a shunt device, wherein the cracking pressure is greater than or equal to 1 mmHg. In some implementations, the techniques described herein relate to a shunt device, wherein the cracking pressure is less than or equal to 20 mmHg. In some implementations, the techniques described herein relate to a shunt device, wherein the leaflet support structure is made of a biocompatible metal alloy or engineering plastics that is configured to deflect in response to the pressure gradient imposed on the leaflet attached to the leaflet support structure. In some implementations, the techniques described herein relate to a shunt device, wherein the leaflet support structure is configured to partially open responsive to the pressure gradient imposed on the leaflet exceeding a first threshold pressure gradient. In some implementations, the techniques described herein relate to a shunt device, wherein the leaflet support structure is configured to fully open responsive to the pressure gradient imposed on the leaflet exceeding a second threshold pressure gradient that is greater than the first threshold pressure gradient. In some implementations, the techniques described herein relate to a shunt device, wherein the leaflet support structure is configured to close responsive to the pressure gradient imposed on the leaflet being less than or equal to the first threshold pressure gradient. In some implementations, the techniques described herein relate to a shunt device, wherein a position of the leaflet support structure between a closed position and a fully open position is related to the pressure gradient imposed on the leaflet, the pressure gradient greater than the first threshold pressure gradient and less than the second threshold pressure gradient. In some implementations, the techniques described herein relate to a shunt device, wherein the shunt device is configured for placement between pulmonary circulation vessels and venous vessels.

In some implementations, the techniques described herein relate to a method for deploying and adjusting a shunt device with an adjustable orifice, the method including: implanting a shunt device in a targeted location in a patient, the shunt device implanted in a crimped configuration, the shunt device including a structural support frame forming an orifice; expanding the shunt device so that the orifice has a first diameter; monitoring patient hemodynamics; and adjusting a diameter of the shunt device to achieve a target flow rate through the orifice, the adjusted diameter being a second diameter different from the first diameter.

In some implementations, the techniques described herein relate to a method, further including adjusting the diameter of the shunt device to a third diameter different from the first diameter and the second diameter responsive to measurements of patient hemodynamics indicating that the flow rate through the orifice is different from a targeted flow rate. In some implementations, the techniques described herein relate to a method, wherein expanding the shunt device includes using a balloon catheter to mechanically expand the shunt device and adjusting the diameter of the shunt device includes using the balloon catheter to deliver heat to the shunt device to cause a shape memory alloy to change shape. In some implementations, the techniques described herein relate to a method, wherein adjusting to the third diameter includes using the balloon catheter that delivered the heat to the shunt device to mechanically expand the shunt device. In some implementations, the techniques described herein relate to a method, wherein the shunt device includes a homogeneous shape memory alloy that is configured to operate in its martensitic phase at body temperature. In some implementations, the techniques described herein relate to a method, wherein the shunt device includes a structural support frame having a nominally uniform chemical composition that is selectively heat treated at a plurality of locations to alter local properties between martensitic and austenitic phases at body temperature. In some implementations, the techniques described herein relate to a method, wherein the shunt device includes a locally variable chemical stoichiometry such that portions of the shunt device operate in a martensitic phase at body temperature and different portions of the shunt device operate in an austenitic phase at body temperature. In some implementations, the techniques described herein relate to a method, wherein the locally variable chemical stoichiometry is achieved by selectively depositing material on portions of the shunt device. In some implementations, the techniques described herein relate to a method, wherein the shunt device further includes: a balloon-expandable structural support frame including metallic alloys; and a super-elastic anchor that provides fixation of the shunt device into the targeted location. In some implementations, the techniques described herein relate to a method, wherein the balloon-expandable structural support frame is integrated with the super-elastic anchor.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the disclosure. In addition, various features of different disclosed embodiments can be combined to form additional embodiments, which are part of this disclosure. Throughout the drawings, reference numbers may be reused to indicate correspondence between reference elements.

FIG. 1 illustrates a cutaway view of the human heart.

FIG. 2 illustrates several access pathways for maneuvering guidewires and catheters in and around the heart to deploy the devices disclosed herein.

FIGS. 3A and 3B illustrate an example shunt device with a check valve configured to open at a threshold pressure and to remain closed below the threshold pressure.

FIGS. 4A, 4B, 4C, and 4D illustrate another example shunt device with a check valve configured to open at a threshold pressure and to remain closed below the threshold pressure.

FIGS. 5A, 5B, 5C, 5D, 5E illustrate another example shunt device with a check valve configured to open at a threshold pressure and to remain closed below the threshold pressure.

FIGS. 5F, 5G, and 5H illustrate that the shunt device of FIGS. 5A-5E regulates flow through the shunt device by balancing the pressure gradient across the shunt device with the leaflet support structure and the leaflet.

FIG. 6 illustrates a flow chart of an example method for deploying and adjusting a shunt device.

FIG. 7 illustrates an example stent device that can be used in the method of FIG. 6.

FIGS. 8A, 8B, and 8C illustrate an example shunt device configured for super-elastic plastic deformation and re-expansion.

FIGS. 9A and 9B illustrate the shunt device of FIGS. 8A-8C with anchors extending from the structural support frame.

FIGS. 10A and 10B illustrate an example shunt device that includes a compact stent for high crimping strain.

FIGS. 11A, 11B, and 11C illustrate the shunt device of FIGS. 10A and 10B with anchors extending from the structural support frame.

FIGS. 12A, 12B, and 12C illustrate a shunt device that includes a pancake stent design formed by a structural support frame.

FIGS. 13A, 13B, 13C, and 13D illustrate deployment of the shunt device of FIGS. 12A-12C.

FIG. 14 illustrates an example stent device that is part of the disclosed variable orifice flow control systems.

FIGS. 15A, 15B, 15C, and 15D illustrate an example of reducing the size of the passageway through an example stent device.

FIG. 16 illustrates an example variable orifice flow control system that includes a control device to deliver energy to a stent device to change the size of the stent device.

### DETAILED DESCRIPTION

The headings provided herein are for convenience only and do not necessarily affect the scope or meaning of the claimed inventive subject matter.

### Overview

Pulmonary hypertension (PH) is a class of ailments involving high blood pressure and it affects the arteries in the lungs and the right side of the heart. PH inflicts at least 2.7 million Americans and complicates common diseases and leads to worsening symptoms and increased mortality. The increased pressure in the blood vessels of the lungs means that the heart has to work harder to pump blood into the lungs. To meet this increased demand, the sympathetic nervous system may recruit more blood from the splanchnic system. Patients with PH are particularly sensitive to these elevated activity levels. A result from the recruitment of blood from the splanchnic system may be pulmonary congestion, which often leads to hospital admission. In particular, certain patients (e.g., those with pulmonary hypertension (PH), including those with pulmonary arterial hypertension (PAH) (e.g., WHO Group 1) or combined pre- and post-capillary pulmonary hypertension (Cpc-PH) due to left heart failure (e.g., WHO Group 2)) may be susceptible to right ventricular afterload or pulmonary congestion.

Preventing or reducing right ventricular afterload and/or alleviating pulmonary congestion (e.g., for congestive heart failure patients) improves outcomes and reduces hospitalizations, morbidity, and/or mortality. A method to prevent or reduce right ventricular afterload is to reduce peak systolic pressure of the right ventricle. Ideally, this reduction comes without compromising the primary right ventricle ejection. Similarly, it is beneficial to reduce systolic and/or mean pulmonary artery pressure and/or mean left arterial pressure while reducing or minimizing the impact on right ventricular function.

Accordingly, described herein are shunts and devices configured to achieve a targeted reduction in peak systolic pressure by percutaneously shunting from the superior vena cava to the right pulmonary artery. The reduction in pressure depends at least in part on a relationship between the cross-sectional area and length of the shunt because this relationship determines the flow rate and pressure drop across the shunt. The targeted reduction in pulmonary pressure can be configured to not exceed the minimum transpulmonary gradient required to maintain forward pulmonary blood flow, taking into account the variability in left atrial pressures (e.g., heart failure or in response to a stimulus). Hence, a shunt that maintains a lower bound may be advantageous, noting that the offset in pressure drop during increases in venous return may not be linear. The shunts disclosed herein can be configured to preserve a minimal transpulmonary pressure while leveraging the pulmonary-caval pressure gradient to offload and reduce systolic and mean pulmonary artery pressure without increasing right ventricle work due to increased venous return and stroke volume.

Also described herein are flow diversion devices placed between pulmonary circulation vessels and venous vessels. The adjustable flow rate devices can be configured to effectively reduce systolic and/or mean pulmonary artery pressure and/or mean left arterial pressure while reducing the impact on the right ventricular function. The disclosed flow diversion devices can be configured to be physiologically responsive to achieve superior hemodynamic response. This provides a number of advantages over fixed-orifice shunt devices that are placed between the left atrium and the right atrium. The disclosed adjustable flow rate devices can also be placed in the interatrial septum or between the left atrium and coronary sinus to divert flow imbalance. In certain instances, after shunt therapy with a flow diversion device inserted between the left atrium and the right atrium to alleviate pulmonary congestion, patient hemodynamics may adjust to the new circulatory circuit condition. The shunt flow may be either too restrictive or not sufficient. In either case, it may be advantageous to modify the desired shunt flow. The disclosed flow diversion devices can be configured to have an adjustable orifice that can be adjusted after initial implantation through a non-invasive or minimally invasive procedure. For example, the disclosed flow diversion devices include stents that are configured to have modifiable diameters. The disclosed flow diversion devices can be implanted and expanded to a first operating diameter and then later activated or adjusted to a second operating diameter.

Furthermore, disclosed herein are devices and systems that are configured to reduce recruitment of blood from the splanchnic system during acutely increased cardiac demand to alleviate pulmonary congestion symptoms, thereby reducing hospital readmission. Disclosed systems include a variable orifice constrictor (e.g., a covered stent) and a controller that delivers energy to the constrictor to reduce the cross-sectional area of the lumen through the variable orifice constrictor. This reduction in cross-section slows down the recruitment of venous blood into the stressed blood volume whereas under normal physiological conditions, exercise or other stimulation would increase the blood volume of the patient which may cause pulmonary congestion.

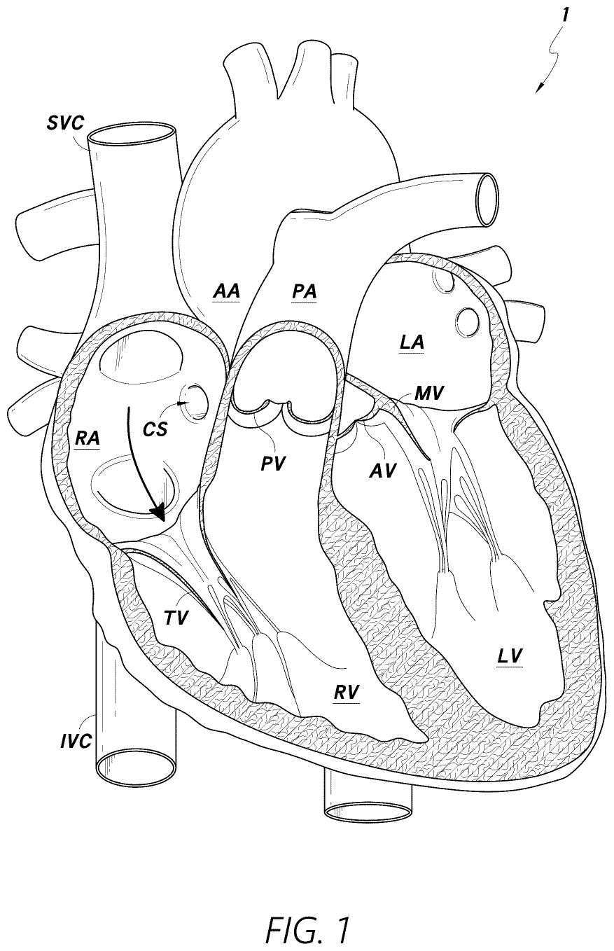

FIG. 1 illustrates a cutaway view of the human heart H. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets extending inward across the respective orifices that come together or “coapt” in the flow stream to form the one-way, fluid-occluding surfaces.

The left atrium LA receives oxygenated blood from the lungs. During the diastolic phase, or diastole, the blood that was previously collected in the left atrium LA (during the systolic phase) moves through the mitral valve MV and into the left ventricle LV by expansion of the left ventricle LV. In the systolic phase, or systole, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein.

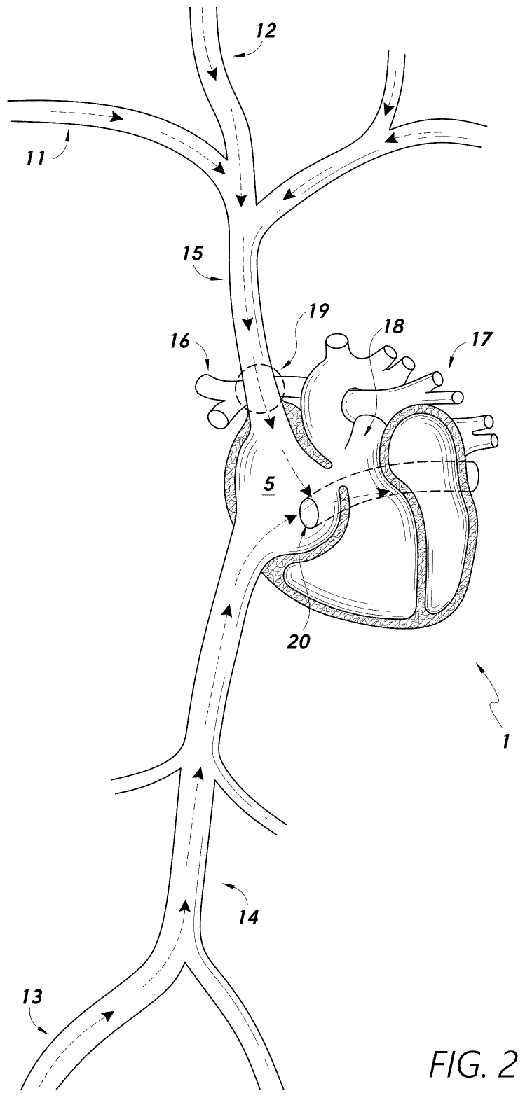

FIG. 2 illustrates several access pathways for maneuvering guidewires and catheters in and around the heart 1 to deploy the devices disclosed herein. For instance, access may be from above via either the subclavian vein 11 or jugular vein 12 into the superior vena cava (SVC) 15, right atrium (RA) 5 and from there into the coronary sinus (CS) 20. Alternatively, the access path may start in the femoral vein 13 and through the inferior vena cava (IVC) 14 into the heart 1. Other access routes may also be used, such as through the pulmonary artery 18, with each access route typically utilizing a percutaneous incision through which the guidewire and catheter are inserted into the vasculature, normally through a sealed introducer. From there, the physician controls the distal ends of the devices from outside the body.

The pulmonary artery 18 branches into a right pulmonary artery (RPA) 16 and a left pulmonary artery (LPA) 17. Some examples of the present disclosure may involve delivering one or more implants (e.g., shunts) to an intersection 19 and/or crossing point between the RPA 16 and the SVC 15. For example, the RPA 16 may extend generally perpendicularly to the SVC 15 and/or can cross behind/in front of the SVC 15. In some cases, the RPA 16 may contact the SVC 15 while in other cases there may be a separation between the RPA 16 and the SVC 15.

Some examples described herein involve delivering a shunt system percutaneously to connect the RPA 16 with the SVC 15. Given that the RPA 16 and SVC 15 are adjacent anatomically, the intersection 19 area of the two provides a desirable location to establish a shunt. Further, because the RPA 16 has higher pressures than the SVC 15, particularly under pulmonary hypertensive conditions, unidirectional movement of blood flow is consistently diverted out of the RPA 16 and into the SVC 15. The net result of this shunting is to decompress and lower the pressure in the main pulmonary artery 18, including mean and peak systolic pressure. This in turn reduces the afterload on the right ventricle and reduces the amount of work required to eject blood, thereby decreasing right ventricle compensatory responses to pulmonary hypertension and preserving ventricular-vascular coupling. Shunting may occur any pressure, or a shunt system may be preloaded to dynamically shunt at an offset or threshold pressure.

Some examples described herein involve delivering a variable orifice flow device percutaneously to the IVC 14 to implant the device in the IVC 14. The variable orifice flow device can be controlled by a system that incorporates physiological feedback (e.g., using one or more pressure sensors). The control system can change the size of the lumen through the variable orifice flow device to reduce blood flow through the device. This can be done to reduce recruitment of blood from the splanchnic system. As a result, pulmonary congestion can be reduced or prevented.

### Example Shunt Devices With Pressure Threshold Valves

As described herein, it is desirable to reduce right ventricular afterload and/or to alleviate pulmonary congestion in patients with pulmonary hypertension or congestive heart failure. The disclosed shunt devices are thus configured to alleviate symptoms and reduce hospital readmission. In particular, for patients with combined pre- and post-capillary pulmonary hypertension due to left heart failure who are non-responsive to vasodilator challenges, it may be particularly desirable to reduce right ventricular load to decrease progression to symptomatic severe right ventricular failure.

Accordingly, disclosed herein are shunt devices that can be placed at junction of the right pulmonary artery (RPA) and the superior vena cava (SVC) to effectively reduce systolic and/or mean pulmonary artery pressure in patients with group 2 pulmonary hypertension, while reducing or minimizing the impact on the right ventricular function. The disclosed shunt devices may also be placed between pulmonary circulation vessels and venous vessels to effectively reduce systolic, mean pulmonary artery pressure, and/or mean left atrial pressure while reducing the impact on the right ventricular function. The disclosed shunt devices include a valve that is configured to open at a threshold pressure differential. The disclosed shunt devices provide advantages over fixed orifice shunt devices that are typically placed between the LA and the RA to alleviate pulmonary congestion. For example, patients with elevated pulmonary resistance may still experience high RV after load at peak systole which may cause RV stress elevation, even in the presence of fixed orifice shunt devices. The disclosed shunt devices include a check valve that provides a variable orifice at the junction of the pulmonary artery and superior vena cava to reduce peak systolic pressure of the RV, without compromising the primary RV ejection. The disclosed shunt devices may also be placed in the interatrial septum to shunt between the LA and the RA with a variable orifice shunt to alleviate pulmonary congestion while maintaining right ventricular function. In some implementations, the disclosed shunt devices can be placed to shunt between the LA and the CS to divert flow imbalances. The disclosed shunt devices can be delivered using a transcatheter delivery system either trans-carotid through the superior vena cava or transfemoral through the inferior vena cava.

The disclosed shunt devices comprise a valved shunt that is preloaded to a target force level during assembly of the shunt device so that the valve is configured to open when a pressure differential exceeds a threshold pressure and the valve is configured to be closed when the pressure differential is at or below the threshold pressure. This may be referred to as a cracking pressure. Cracking pressure is the minimum upstream pressure required to open a check valve enough to allow detectable flow. Detectable flow is when the check valve allows a small but steady flow of liquid or gas to pass through the valve body and out through its outlet port. In other words, check valve cracking pressure is a measure of the pressure differential between the inlet and outlet ports of the valve when flow is first detected.

The disclosed shunt devices can be configured with a cracking pressure that is configured to not exceed the minimum transpulmonary gradient required to maintain forward pulmonary blood flow, taking into account the variability in left atrial pressures. Advantageously, the disclosed shunt devices can be configured to maintain a lower pressure bound to preserve a minimal transpulmonary pressure while leveraging the pulmonary-caval pressure gradient to offload and reduce systolic and mean pulmonary artery pressure without increasing right ventricle work due to increased venous return and stroke volume.

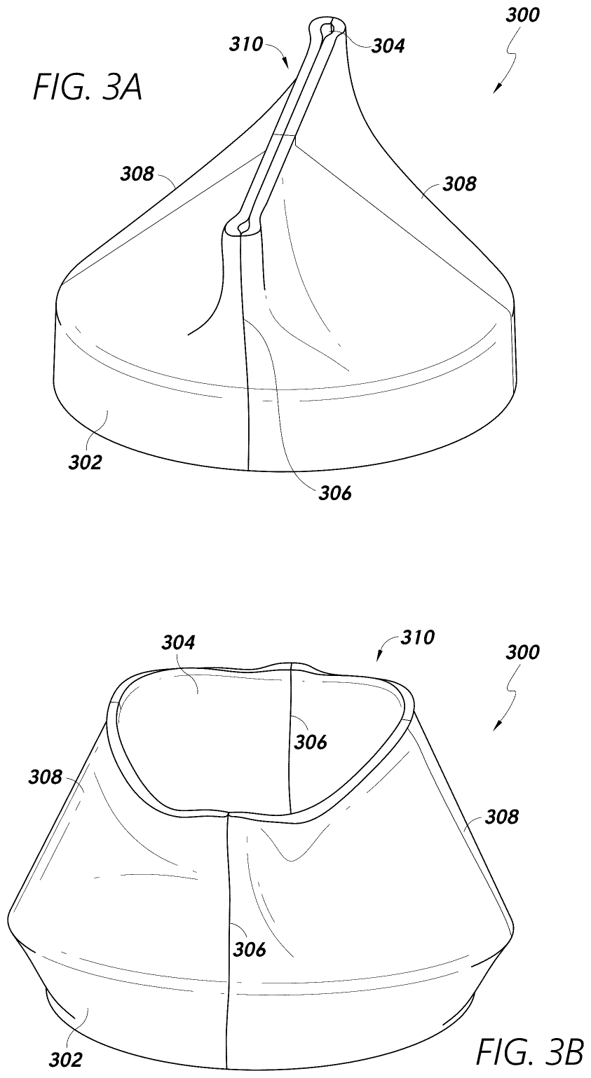

FIGS. 3A and 3B illustrate an example shunt device 300 with a check valve 310 configured to open at a threshold pressure and to remain closed below the threshold pressure. The shunt device 300 includes an inlet port 302 and an outlet port 304 formed by the check valve 310. The check valve 310 can be constructed of metallic alloys or plastics. The shunt device 300 includes at least two commissure posts 306 that support the attachment of tissue leaflets 308. The leaflets 308 form the outlet port 304 and are configured to open and close responsive to a pressure differential between the inlet port 302 and the outlet port 304.

The shunt device 300 can be preloaded to a target force level while the leaflet assembly 306, 308 is preformed such that the leaflets 308 are naturally closed (as shown in FIG. 3A) responsive to a pressure differential between the inlet port 302 and the outlet port 304 being less than or equal to a threshold pressure. The leaflets 308 are configured to open (as shown in FIG. 3B) responsive to the pressure differential being greater than the threshold pressure. In some implementations, the threshold pressure is zero. In some embodiments, the pressure differential is about 20 mmHg.

The leaflets 308 are hydraulically loaded to deform the commissure posts 306 such that the leaflets 308 open (as shown in FIG. 3B) to form an open orifice that allows blood flow. The leaflets 308 can be configured to close when the pressure differential drops below the set pressure or zero. The shunt device 300 is configured to remain closed or to have a small opening at low pressure differentials (e.g., less than a set pressure of about 20 mmHg) and/or during early right ventricle systole. At the mid-systole of the right ventricle when the pulmonary arterial pressure increases (e.g., exceeds about 20 mmHg), the shunt device 300 is configured to deform to allow the leaflets 308 to open to create a shunt flow (e.g., from the pulmonary artery to the superior vena cava). The check valve 310 can then reduce the orifice dimension (e.g., return to the closed position) responsive to the pulmonary artery pressure dropping below about 20 mmHg. In this fashion, the pulmonary artery pressure can rise and fall in response to right ventricle contraction and relaxation but can remain within a small range of the set pressure of about 20 mmHg. In this way, right ventricle overload may be avoided. The set pressure of 20 mmHg is used as an example for illustrative purposes. However, this pressure can be adjusted through different design variations. The shunt device 300 can be tailored to meet specific patient needs.

Accordingly, disclosed herein are shunt devices configured to alleviate pressure overload of a heart organ. The shunt device includes an inlet port and an outlet port. The shunt device includes two or more leaflets to form the outlet port, the two or more leaflets pre-formed to hold a closed state at or under a threshold pressure differential, the leaflets configured to open to allow blood flow through the shunt device in response to a pressure that exceeds the threshold pressure differential. The shunt device includes two or more commissure posts to couple the leaflets to the shunt device. The shunt device may at least partially be composed of metallic alloys or plastics. The shunt device is configured for placement at the SVC/PA junction. The shunt device is configured to alleviate right ventricle pressure. The shunt device is configured to remain closed when the pressure differential between the inlet and outlet ports is at or below about 20 mmHg and to open responsive to the pressure differential exceeding 20 mmHg.

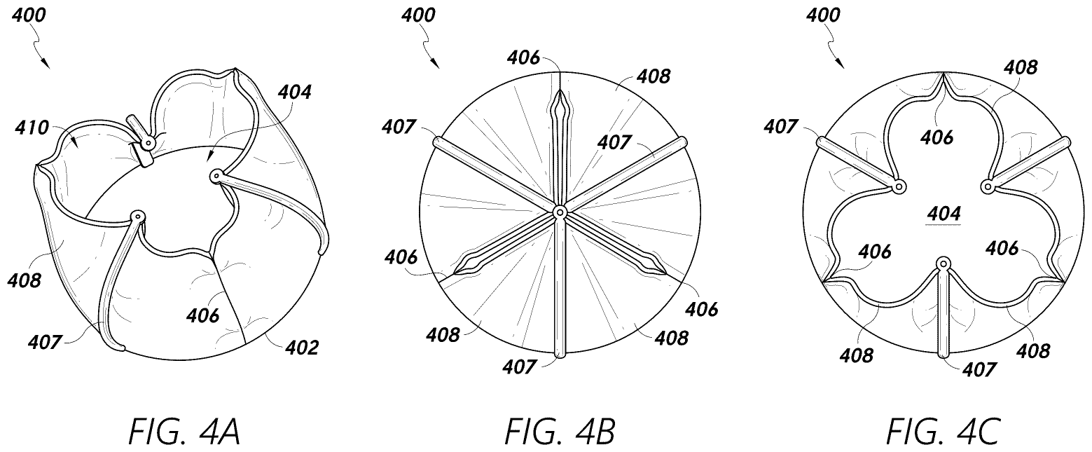

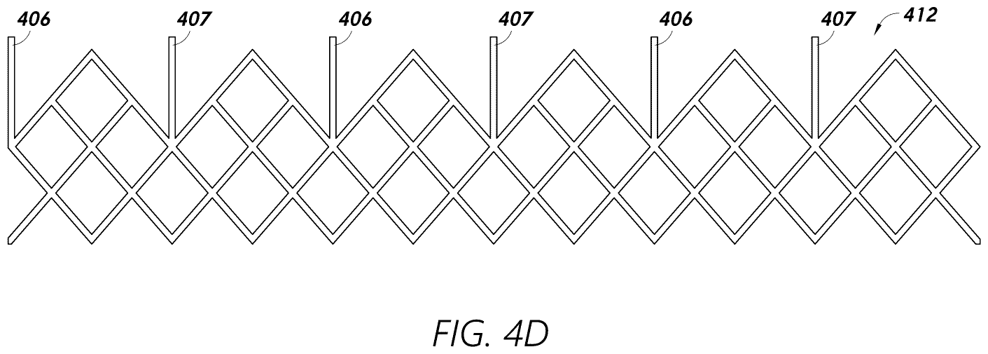

FIGS. 4A, 4B, 4C, and 4D illustrate another example shunt device 400 with a check valve 410 configured to open at a threshold pressure and to remain closed below the threshold pressure. The shunt device 400 includes an inlet port 402 and an outlet port 404 formed by the check valve 410. The check valve 410 can be constructed of metallic alloys or plastics. The shunt device 400 includes at least three commissure posts 406 that support the attachment of tissue leaflets 408 and at least three cusp posts 407 that regulate the opening of the leaflets 408. The leaflets 408 form the outlet port 404 and are configured to open and close responsive to a pressure differential between the inlet port 402 and the outlet port 404.

The shunt device 400 can be preloaded to a target force level while the leaflet assembly 406, 408 is preformed such that the leaflets 408 are naturally closed (as shown in FIG. 4B) responsive to a pressure differential between the inlet port 402 and the outlet port 404 being less than or equal to a threshold pressure. The leaflets 408 are configured to open (as shown in FIG. 4C) responsive to the pressure differential being greater than the threshold pressure. In some implementations, the threshold pressure is zero. In some embodiments, the pressure differential is about 20 mmHg.

The leaflets 408 are hydraulically loaded to deform the commissure posts 406 such that the leaflets 408 open (as shown in FIG. 4C) to form an open orifice that allows blood flow. The leaflets 408 can be configured to close when the pressure differential drops below the set pressure or zero. The shunt device 400 is configured to remain closed or to have a small opening at low pressure differentials (e.g., less than a set pressure of about 20 mmHg) and/or during early right ventricle systole. At the mid-systole of the right ventricle when the pulmonary arterial pressure increases (e.g., exceeds about 20 mmHg), the shunt device 400 is configured to deform to allow the leaflets 408 to open to create a shunt flow (e.g., from the pulmonary artery to the superior vena cava). The check valve 410 can then reduce the orifice dimension (e.g., return to the closed position) responsive to the pulmonary artery pressure dropping below the set pressure (e.g., about 20 mmHg). In this fashion, the pulmonary artery pressure can rise and fall in response to right ventricle contraction and relaxation but can remain within a small range of the set pressure (e.g., about 20 mmHg). In this way, right ventricle overload may be avoided. The set pressure of 20 mmHg is used as an example for illustrative purposes. However, this pressure can be adjusted through different design variations. The shunt device 400 can be tailored to meet specific patient needs. The shunt device 400 may also be referred to as a flow diversion device.

FIG. 4D illustrates that the shunt device 400 can include a structural support frame 412. The structural support frame 412 can be made of biocompatible metal alloy or engineering plastics. The structural support frame 412 is configured to deflect in response to a hydraulic pressure gradient imposed on the leaflets 408 attached to this structural support frame 412. The leaflets 408 comprise a plurality of flexible membranes that form a flow orifice or outlet port 404. The leaflets 408 are configured to respond to flow pressure to change the orifice area (e.g., the size of the outlet port 404). The structural support frame 412 can be made to form a tri-leaflet variable orifice support with three stiff posts 406 and three flexible posts 407. The flexible posts 407 are configured to cause the leaflets 408 to be closed when a pressure differential is at or below a threshold pressure differential and are configured to deflect under a pressure differential on the leaflet inflow surface that exceeds the threshold pressure differential. The leaflets 408 can be made from suitable biocompatible material, such as animal tissue including bovine pericardial tissue, equine pericardial tissue, porcine pericardial tissue, and other thin tissue. The leaflets 408 can be made from biocompatible polymers.

Thus, in some implementations, the disclosed shunt device 400 comprises a pressure-sensitive variable orifice flow diversion device configured to open in response to increased pressure to shunt blood between pulmonary circulation vessels and venous vessels. The variable orifice shunt device 400 may be deployed at different anatomic locations to alleviate pressure overload of the relevant end organ, such as PA-LA, interatrial septum, and LA-CS.

Accordingly, disclosed herein are shunt devices configured to alleviate pressure overload of a heart organ. The shunt device includes an inlet port and an outlet port. The shunt device includes a check valve formed from three or more leaflets that are pre-formed to hold a closed state or to have a smaller opening (which may be preset or adjustable) under zero or minimal pressure and configured to open to allow blood flow through the shunt device in response to increased pressure. The shunt device includes at least three commissure posts that support the attachment of leaflets to the shunt device. The shunt device includes at least three cusp posts that regulate opening of the leaflets. The shunt device may be at least partially composed of metallic alloys or plastics. The shunt device may be configured for placement at various anatomical locations that include PA-LA, PA-SVC, interatrial septum, and LA-CS). The shunt device may be a variable orifice shunt device. The shunt device may be a variable flow control device (e.g., a device that provides a plurality of flow states. The shunt device may be configured to alleviate right ventricle pressure. The shunt device may be configured to remain closed or to have a small preset opening at or below a threshold pressure differential (e.g., about 20 mmHg) between the inlet port and the outlet port and to open (e.g., the outlet port being larger than the preset opening size) at a pressure differential above the threshold pressure differential (e.g., about 20 mmHg). The outlet port can be configured to form a shamrock shape in the open configuration. The commissure posts and cusp posts are configured to regulate the action of the leaflets and/or to define the shape and size of the orifice. The shunt device may include a structural support frame. The structural support frame can be made to form a tri-leaflet variable orifice support with three stiff posts corresponding to the three or more commissure posts and three flexible posts corresponding to the three or more cusp posts.

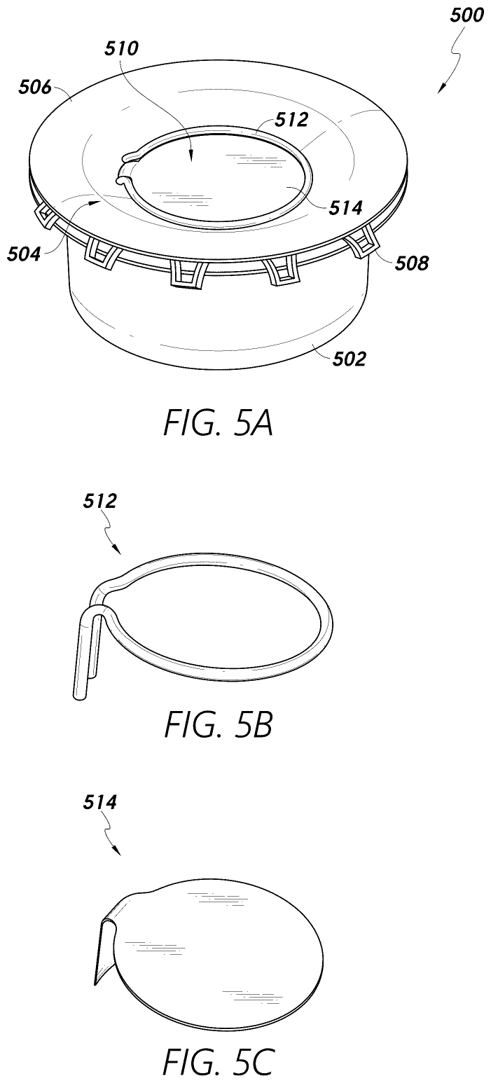

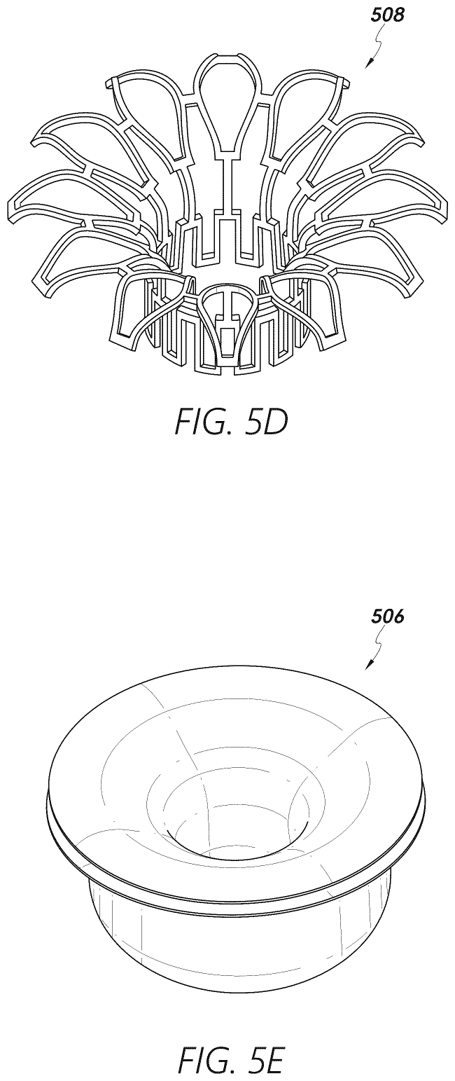

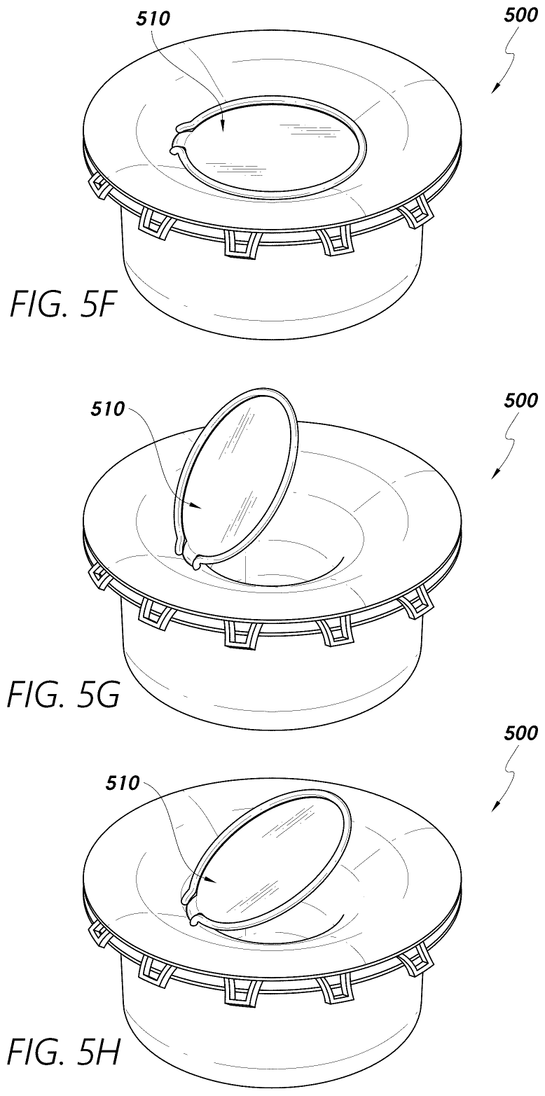

FIG. 5A illustrates another example shunt device 500 with a check valve 510 configured to open at a threshold pressure and to remain closed below the threshold pressure. The shunt device 500 is further configured to partially open responsive to a pressure differential, the size of the opening of the check valve 510 being related to the pressure differential. For example, in the partially open configuration, an increase in the pressure differential causes the check valve 510 to open more until it reaches a fully open configuration. Likewise, in the fully open or partially open configuration, a decrease in the pressure differential causes the check valve 510 to close more until it reaches a closed configuration (e.g., when the pressure differential falls below a pressure differential threshold). Thus, the shunt device 500 is physiologically responsive. The shunt device 500 can be placed between pulmonary circulation vessels and venous vessels. The shunt device 500 can effectively reduce systolic, mean pulmonary artery pressure, and/or mean left atrial pressure while reducing or minimizing the impact on right ventricular function. The shunt device 500 can be advantageously placed in the interatrial septum, the wall between the left atrium and the coronary sinus, or the pulmonary artery (PA)-superior vena cava (SVC) junction. However, the shunt device 500 can be placed in any suitable vessel or between suitable vessels to provide a variable orifice flow control device.

The shunt device 500 includes an inlet port 502 and an outlet port 504 wherein flow through the shunt device 500 is regulated by the check valve 510. The check valve 510 can be described as a single flapper check valve. The shunt device 500 includes an elastic or super-elastic leaflet support structure 512 (e.g., a leaflet support wireform) as illustrated in FIG. 5B, a leaflet 514 (e.g., a soft tissue or polymer membrane) supported by the leaflet support structure 512 that together form the preloaded check valve 510, the leaflet 514 illustrated in FIG. 5C, a shunt frame 508 (e.g., a structural lumen support) as illustrated in FIG. 5D, and a wrapping membrane 506 (e.g., a sealing cloth or soft tissue wrap) as illustrated in FIG. 5E. FIG. 5A illustrates the shunt device 500 in an assembled configuration.

The shunt device 500 includes a single flapper check valve 510. The shunt frame 508 forms a shunt-like support structure to shunt an opening between circulatory systems (e.g., PA-SVC junction, LA-RA septum, LA-CS wall). The wrapping membrane 506 wraps the shunt frame 508 to form a sealing face. The leaflet support structure 512 forms a single-looped elastic or super-elastic structure to support the flapper leaflet 514. The leaflet 514, supported by the leaflet support structure 512, regulates the flow at the check valve 510. In some examples, the leaflet support structure 512 can be made from an elastic material or a super-elastic alloy so that the cracking pressure of the check valve can be set to a targeted value. In some examples, the targeted value of the cracking pressure is between about 1 mmHg and about 20 mmHg.

The leaflet 514 can be made from suitable biocompatible properly fixed animal tissue such as bovine pericardial tissue, equine pericardial tissue, porcine pericardial tissue, and other thin tissue. The leaflet 514 can be made from biocompatible polymers.

In some examples, the shunt device 500 can be configured such that the check valve 510 remains closed or has a small opening at a pressure gradient less than a threshold pressure (e.g., 20 mmHg). This may occur, for example, during early right ventricular systole. At the mid-systole of the right ventricle, when the pulmonary arterial pressure is sufficient to cause a pressure differential across the shunt device 500 that exceeds the threshold pressure differential, the check valve 510 is configured to at least partially open to create a shunt flow (e.g., from the PA to the SVC). The check valve 510 is configured to close responsive to the pressure differential dropping below the threshold pressure differential. In this fashion, the PA pressure can rise and fall in response to the RV contraction and relaxation but remains within a small range of the targeted pressure (e.g., 20 mmHg). This can be done to prevent or reduce RV overload. The targeted pressure can be tailored using different design variations and can be tuned or optimized based on clinical testing. For example, it may be desirable to create different variations of the disclosed shunt device 500 to fit specific patient needs.

The shunt device 500 can be placed between pulmonary circulation vessels and venous vessels to divert flow from pulmonary artery or pulmonary vein or left atria to the venous vessels (SVC, right atrium, coronary sinus), thereby effectively reducing systolic and/or mean pulmonary artery pressure and/or mean left atrial pressure, while reducing or minimizing the impact on the right ventricular function. The shunt device 500 is a variable orifice device placed at the junction between pulmonary artery and superior vena cava in anatomically suitable patient to create a flow diversion shunt pathway, thereby reducing peak systolic pressure of the RV, without compromising the primary RV ejection. The shunt device 500 can be inserted between the LA and the RA to provide a variable orifice flow to alleviate pulmonary congestion while maintaining right ventricular function. The shunt device 500 can be inserted at the interatrial septum or LA-CS wall to divert flow imbalance.

FIGS. 5F, 5G, and 5H illustrate that the shunt device 500 regulates flow through the shunt device 500 by balancing the pressure gradient across the shunt device 500 with the leaflet support structure 512 and the leaflet 514. Accordingly, the shunt device 500 can operate in a number of states. For example, the check valve 510 can be set to be closed (FIG. 5F), preloaded (FIG. 5G), or partially open (FIG. 5H). In some examples, upon imposing a pressure gradient from the LA to the RA, the check valve 510 is configured to open gradually in response to the physiological pressure gradient, allowing more blood to be shunted from pulmonary circulation to systemic circulation return. This advantageously relieves the pulmonary congestion a targeted amount.

Accordingly, the shunt device includes a shunt frame that forms an inlet port and an outlet port and that is configured to support a shunt opening. The shunt device includes a leaflet support structure coupled to the support frame. The shunt device includes a leaflet comprising a soft tissue or polymer membrane, the leaflet coupled to the leaflet support structure. The leaflet support structure is configured to deflect in response to a pressure differential or a pressure gradient imposed on the leaflet. The shunt device includes a wrapping membrane configured to wrap the support frame to regulate a flow rate through the shunt device. The leaflet and the leaflet support structure cooperate to form a flapper check valve that provides a variable orifice flow responsive to the pressure gradient imposed on the leaflet. In some implementations, the leaflet support structure comprises an elastic material or a super-elastic alloy configured so that a cracking pressure of the shunt device can be set to a targeted value (e.g., between 1 mmHg and 20 mmHg). In some implementations, the leaflet support structure is made of a biocompatible metal alloy or engineering plastics that is configured to deflect in response to hydraulic pressure gradient imposed on the leaflet attached to the leaflet support structure.

### Example Shunt Devices With an Adjustable Orifice

Disclosed herein are shunt devices with an adjustable orifice that are configured to have its internal orifice diameter modified at some time post-implantation to at least a second diameter. The disclosed shunt devices can be configured to have the internal orifice diameter modified after implantation one or more times. In operation, these shunt devices are implanted and balloon-expanded or self-expanded to a first diameter. At some point, if it is determined that the diameter is too large, the shunt device can be activated to a reduced diameter state, which can subsequently be re-expanded to a different diameter than the first diameter. The ability to adjust the diameter of the shunt device allows fine-tuning or alteration of its pressure modulating ability.

The disclosed shunt devices can be implanted and expanded to an initial or first diameter. However, after shunt therapy with the implanted shunt device (e.g., implanted between the LA and the RA) initially alleviates pulmonary congestion, patient hemodynamics typically adjust according to the new circulatory circuit condition. The flow through the shunt device may then be either too restrictive or not sufficient. Either way, the disclosed shunt devices include a means for reintervention to modify the flow through the shunt device. In particular, the disclosed shunt devices can be adjusted after initial implantation through either non-invasive or minimally invasive procedures to adjust the size of the orifice of the shunt device.

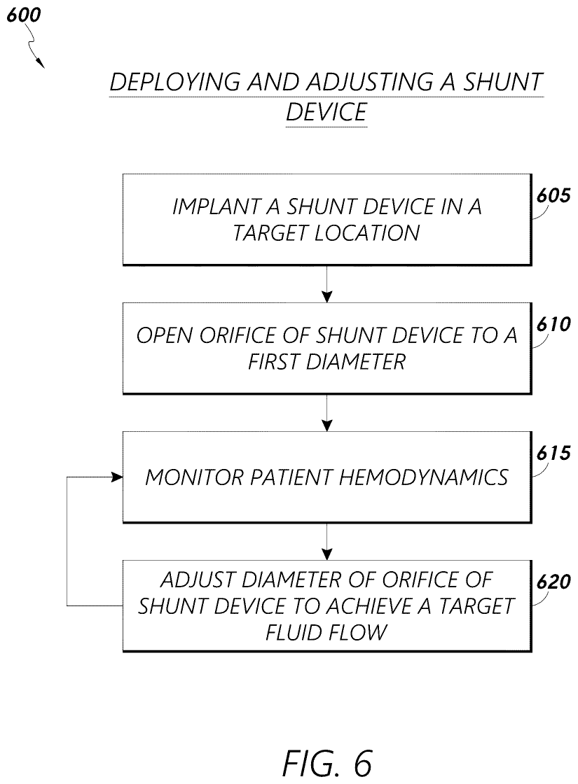

FIG. 6 illustrates a flow chart of an example method 600 for deploying and adjusting a shunt device. The shunt device can be placed between pulmonary circulation vessels and venous vessels to divert flow from the pulmonary artery, pulmonary vein, or left atria to the venous vessels (e.g., the SVC, right atrium, coronary sinus), thereby effectively reducing systolic and/or mean pulmonary artery pressure and/or mean left atrial pressure, while reducing or minimizing the impact on the right ventricular function. In some embodiments, the shunt device can be inserted at the interatrial septum or LA-CS wall to divert flow imbalance.

In block 605, a shunt device is implanted in a targeted location, such as in the interatrial septum between the left atrium and the right atrium. In block 610, the shunt device is expanded to an initial or first diameter to provide shunt flow through the shunt device. In block 615, patient hemodynamics are monitored to determine whether the shunt flow is too high or too low. Patient hemodynamics can be ascertained through common hemodynamics measuring and monitoring systems such as transcatheter pressure/flow sensors or echo flow transducers. Responsive to determining that the shunt flow is outside of a targeted range, in block 620 the diameter of the orifice of the shunt device is adjusted to achieve a shunt flow in the targeted range. The blocks 615 and 620 can be repeated to adjust the diameter of the orifice more than once to achieve the targeted shunt flow.

For example, when deemed beneficial, a balloon catheter can be redeployed to cause the shunt stent device to expand to a new diameter to allow more shunt flow. When necessary, a heating catheter can be deployed to recover the stent geometry to a smaller diameter to reduce the shunt flow. As another example, the stent device can be made of an alloy at least partially in martensitic state at body temperature such that the stent device is constructed in such a way that it may be compressed to a small state for deployment into a patient's anatomy. Once placed, the stent device may be plastically deformed to a larger diameter to achieve a targeted pressure equalization. At a later time, it may be determined that the diameter is too large, and it is desired to reduce the diameter. An RF device may be used to heat the device to its austenite temperature whereby the device reverts to a predetermined size. It may then be re-enlarged to an intermediate diameter, for example, by inflating the same balloon that provided the RF energy to adjust the size.

The shunt devices suitable for use in the method 600 can have a variety of characteristics. For example, in a first example implementation, the shunt device is constructed of a homogeneous shape-memory alloy, for example, NiTi or NiTi-based alloys, whereby the alloy is processed such that the device functions at least partially in its martensitic phase at body temperature (e.g., 37° C.±5° C.). In such a shunt device, the Aₛ temperature, defined as the onset temperature where the alloy reverts from its martensitic phase to its austenitic phase or parent phase as in a single-stage reverse transformation, or the R′ₛ temperature, defined as the onset temperature where the alloy transforms from its martensitic phase to its R phase as in a two-stage reverse transformation in Nitinol, is approximately 37° C. or greater.

In a second example implementation, the shunt device includes a structural support frame having a nominally uniform chemical composition that is selectively heat treated at various locations to alter the local properties between martensitic and austenitic phases at body temperature.

Portions of the shunt device can be subjected to selective heat-treatment or laser-heated to locally modify the transition temperatures of the alloy hence locally modifying the functional properties of the shunt device. Thus, portions of the shunt device can be configured to have relatively high transition temperatures, such as Aₛ at or about 37° C. or greater, while other portions of the shunt device can be configured to have relatively lower transition temperatures, such as A_{f} less than 37° C.

In a third example implementation, the shunt device can be configured to have locally variable chemical stoichiometry (e.g., different Ni:Ti ratio in the example of a NiTi binary alloy) such that portions of the shunt device operate in the martensitic phase while other portions operate in the austenitic phase. One method of providing variable chemical composition is through vapor deposition techniques. By selectively depositing material on portions of the shunt device, those areas can be configured to function in a phase different than other portions of the shunt device that have not had material deposited thereon. Another method of providing variable chemical compositions is through selective evaporation of material from the blank during the wire-frame laser cutting process.

In a fourth example implementation, the shunt device comprises a balloon-expandable device made of CoCr-based alloys, Fe-based stainless-steel alloys, Ti-based alloys, NiTi-based alloys, or other metallic alloys. The shunt device further includes a super-elastic anchor that provides the fixation of the structural support frame into the anatomical destination. The balloon-expandable structural support frame can be integrated with the self-expandable super-elastic anchor by mechanical means such as suturing, mechanical interlocking, or welding.

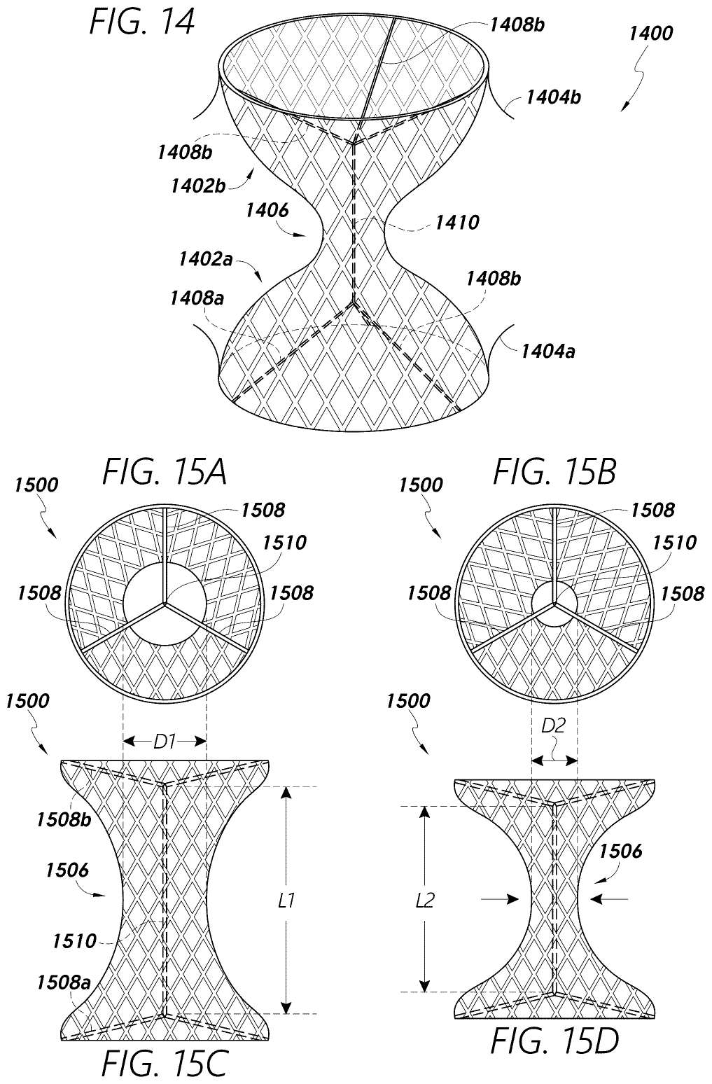

In a fifth example implementation, the shunt device includes an elastic stent-like structure with mechanical actuators that include shape-memory materials, similar to the stent devices described with respect to FIGS. 14 and 15A-D. The shape-memory materials can be thermally activated (e.g., by dielectric heating) or electrically activated (e.g., by an electrical direct current) to increase or reduce the diameter of the stent-like structure (e.g., the diameter of the structural support frame).

The disclosed shunt devices for use in the method 600 can be designed using typical endovascular stent geometry typical of cylindrical revolved profiles to be able to be retrieved through a catheter and deployed in the catheter. The shunt devices can be configured to assume a certain predefined geometry per the designed flow requirement. The shunt devices can then be configured to be adjusted to achieve a range of flow rates through the shunt device.

The disclosed shunt devices include a structural support frame made from re-shapable metal that can be made to change orifice area post deployment through mechanical and electro-thermal means. The structural support frame can be made from martensitic Nitinol with A_{f} temperature greater than about 50° C., in certain implementations. In some implementations, the structural support frame can be made from biocompatible strain hardening alloys such stainless steel and Co—Cr alloys.

The disclosed shunt devices can be configured to be radio-opaque for x-ray visualization of the device size and location. This can be accomplished using PVD heavy element coating and applying masking for localization. The disclosed shunt devices can be configured to shape-change with martensite at or near body temperature, e.g., A_{f}>40° C. The shunt devices can be configured to be plastically deformed using balloon expansion and can be configured to be deformed using shape set materials for deployment. The shunt devices can be configured to be re-expanded post-deployment. In some implementations, the shunt devices have geometries that provide suitable radial strengths, that are crimp-able to 20 Fr, that are radio-opaque, and/or that include a specific stent anchoring mechanism. To be balloon expandable and later adjusted using thermal or electrical means, the disclosed shunt devices can include a martensite portion and an austenite portion, the composite design also including super-elastic and elastic-plastic portions. To adjust the size of certain implementations of the shunt devices, RF energy can be applied using transcatheter means. In some implementations, the shunt device can be made using a single cell stent design. In some implementations, the structural support frame includes a braid structure with composite wires, such as Nitinol wire and CoCr DFT (drawn filled tubing) wire.

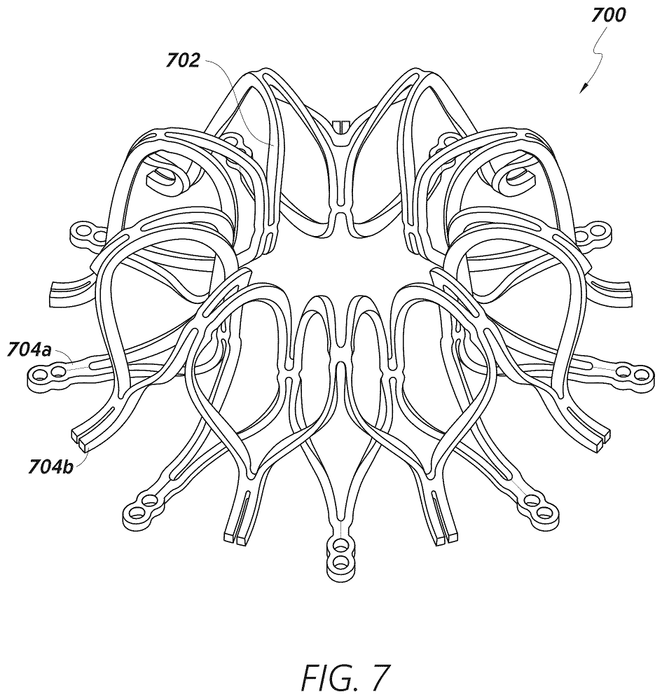

FIGS. 7-13D illustrate example stent devices that can be used in the method 600. These example stent devices represent example embodiments, however, variations of the disclosed stent devices may be utilized without departing from the scope of this disclosure. In addition, the example shunt devices can incorporate one or more of the properties described above with respect to deployment, size adjustment, radio-opacity, material composition, and the like.

FIG. 7 illustrates an example shunt device 700 with a structural support frame 702 configured to anchor the shunt device to tissue by crimping the tissue between upper and lower portions 704a, 704b of the structural support frame. The shunt device 700 can be balloon-expanded to a first diameter. After deployment, the shunt device 700 can be adjusted to decrease or increase the diameter of the orifice formed by the shunt device 700. This can be accomplished using mechanical means, thermal means, or electrical means, as described herein. This can also be accomplished due at least in part to the composition of the structural support frame 702, as described above with respect to the example shunt device embodiments.

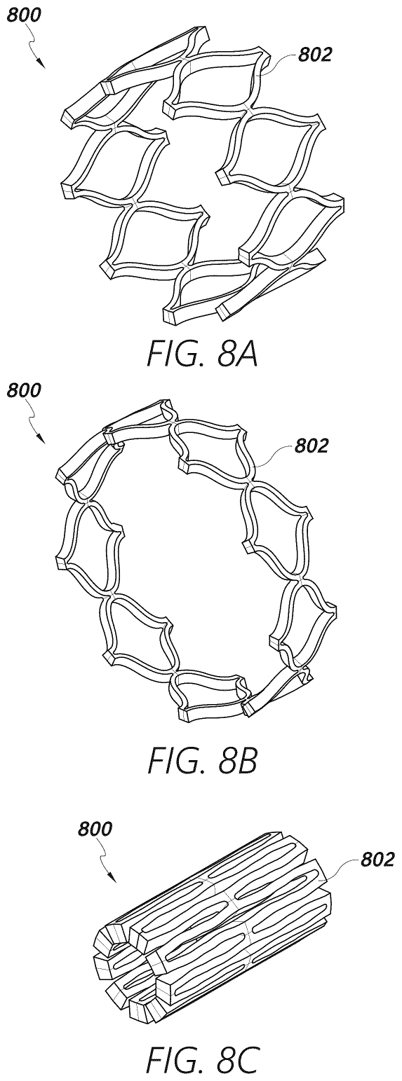

FIGS. 8A, 8B, and 8C illustrate an example shunt device 800 configured for super-elastic plastic deformation and re-expansion. The shunt device 800 can include a structural support frame 802 that is laser cut. The structural support frame 802 can include material configured to transition from a martensite phase to an austenite phase, the material being a portion of the structural support frame or all of the structural support frame 802 and/or the material having a different alloy composition in different locations of the structural support frame.

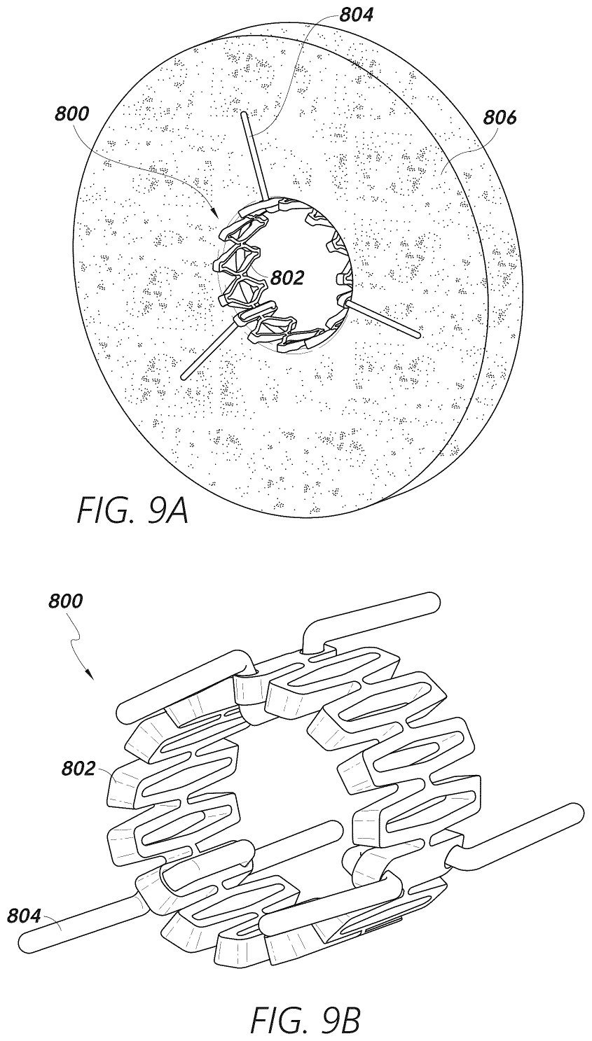

FIGS. 9A and 9B illustrate the shunt device 800 of FIGS. 8A-8C with anchors 804 extending from the structural support frame 802. The anchors 804 can be axially aligned during delivery and deployment (FIG. 9B) and can be transitioned to extend radially from the structural support frame 802 to anchor the shunt device 800 to tissue 806 (FIG. 9A). Thus, the shunt device 800 can include a balloon-expandable stent with a flat anchor 804.

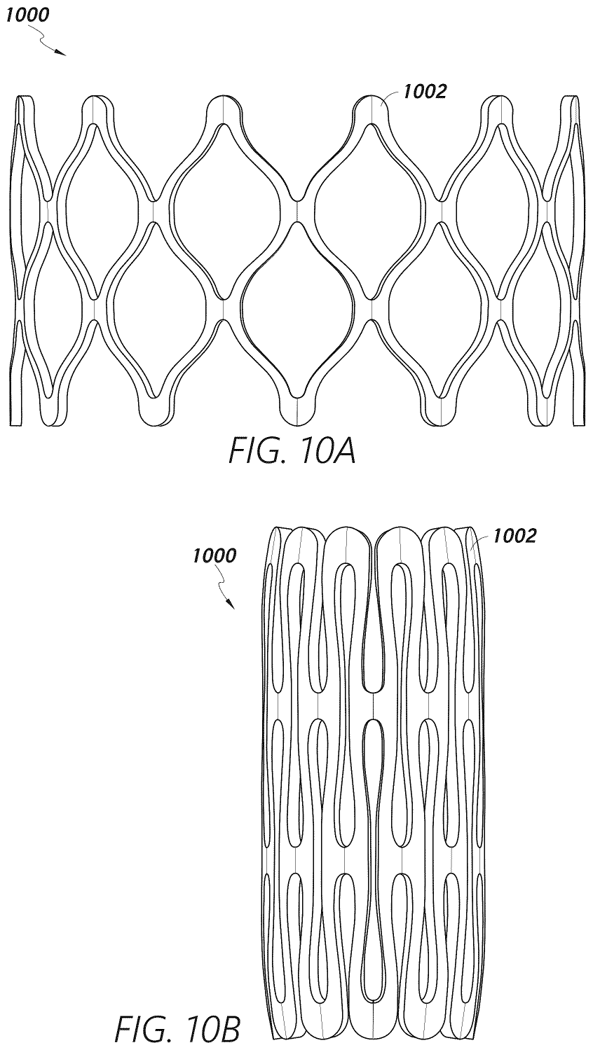

FIGS. 10A and 10B illustrate an example shunt device 1000 that includes a compact stent for high crimping strain at least partially beyond the elastic deformation limit of stress-induced martensite. Such a high deformation results in plastic deformation as a result of retained martensite that is stable at the body temperature. The device having such retained martensite can be further plastically deformed to retain the deformed shape. The shunt device 1000 includes a structural support frame 1002 (e.g., a stent) that is super elastic and that can be oversized after deployment and over-crimped to plastic deformation for delivery. The shunt device 1000 can be configured for post-deployment dilation for resizing, with plastic deformation confined within the cylindrical stent junctions 1002. The strut taper of the structural support frame 1002 can be adjusted and tailored to spread plastic strain in the crimped configuration.

FIGS. 11A, 11B, and 11C illustrate the shunt device 1000 of FIGS. 10A and 10B with anchors 1004 extending from the structural support frame 1002. The anchors 1004 can be axially aligned during delivery and deployment (FIG. 11B) and can be transitioned to extend radially from the structural support frame 1002 to anchor the shunt device 1000 to tissue (FIG. 11C). Thus, the shunt device 1000 can include a balloon-expandable stent with a flat anchor 1004. The shunt device 1000 can be expanded after deployment (FIG. 11A).

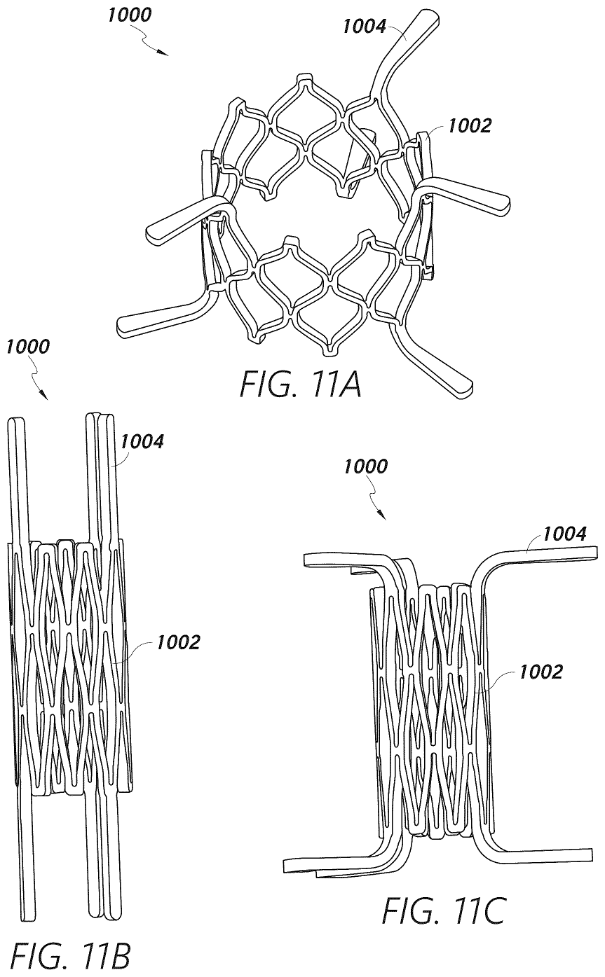

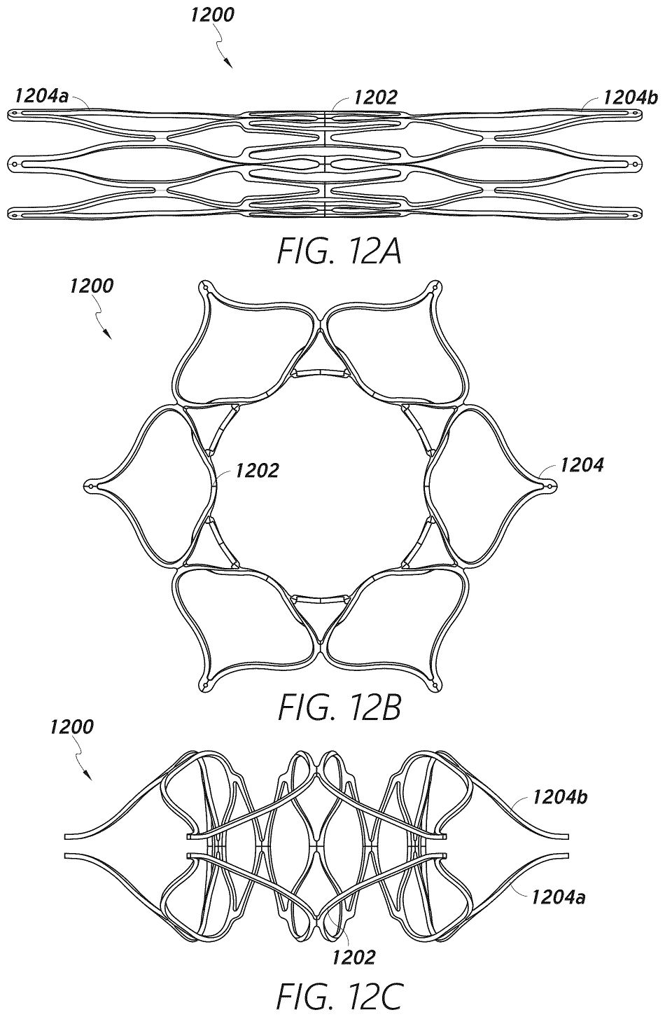

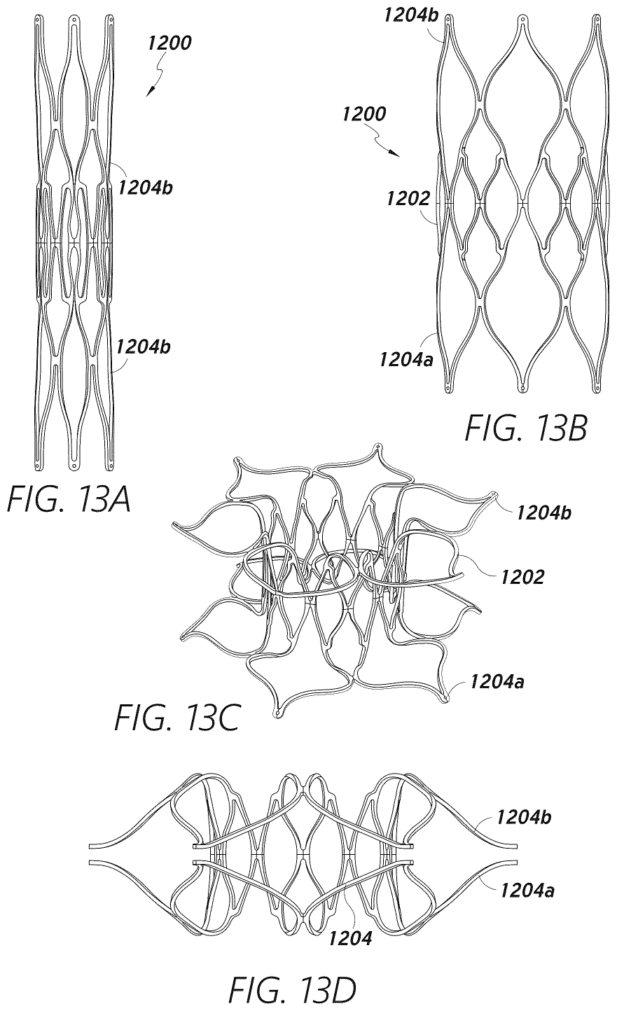

FIGS. 12A, 12B, and 12C illustrate a shunt device 1200 that includes a pancake stent design formed by a structural support frame 1202. The shunt device 1200 is configured to be a balloon-expandable stent for diameter adjustment combined with a super elastic stent for anchoring. FIGS. 13A, 13B, 13C, and 13D illustrate deployment of the shunt device 1200 of FIGS. 12A-12C. The end portions 1204a, 1204b of the shunt device 1200 are configured to fold outward to pinch tissue between the end portions 1204a, 1204b to anchor the shunt device 1200 to tissue. This is similar to the shunt device 700 of FIG. 7.

### Example Variable Orifice Flow Control Systems

To reduce recruitment of blood from the splanchnic system, which may occur during periods of increased cardiac demand, variable orifice flow control systems are described herein. The disclosed systems can reduce blood recruitment from the splanchnic system to alleviate symptoms of pulmonary congestion, thereby reducing hospital readmissions. The disclosed variable orifice flow control systems are configured to provide variable flow control of an implanted variable orifice flow control device (e.g., a covered stent) to provide variable flow control through the device. The disclosed systems are configured to provide adjustable flow rates by implanting a covered stent device in the venous circuit, the covered stent device providing a variable orifice. The size or cross-section of the passageway through the device is controlled using a control system that can modulate the size of the orifice based on physiological measurements (e.g., blood pressure). This can be particularly advantageous to modulate the stressed blood volume. The variable orifice flow control systems include a stent device responsible for changing the area of the orifice and a control device configured to provide energy to the stent device to actuate the orifice structure of the stent device. In some implementations, the variable orifice flow control device (e.g., a stent device) can be delivered via a catheter through the jugular vein into the IVC and the associated control device can be implanted in the patient (e.g., at or near the heart) or on the patient (e.g., external to the patient). The disclosed variable orifice flow control device can be implanted in other vessels where it is desirable to regulate flow through the device using a control system.

FIG. 14 illustrates an example stent device 1400 that is part of the disclosed variable orifice flow control systems. The stent device 1400 can be an endovascular covered stent device. The stent device 1400 includes apposition ends 1402a, 1402b with anchors 1404a, 1404b (e.g., barbs) at each apposition end 1402a, 1402b to secure the stent device 1400 onto the wall of the vessel in which it is implanted. The stent device 1400 includes a reduced orifice formed by a middle, narrowed portion 1406 (or a variable orifice portion) connecting the two apposition ends 1402a, 1402b that is between the two apposition ends 1402a, 1402b. The apposition ends 1402a, 1402b are flared portions that lead into the middle, narrowed portion 1406 or variable orifice portion. The stent device 1400 includes bridge elements 1408a, 1408b at each apposition end 1402a, 1402b to couple an actuating element 1410 to each apposition end 1402a, 1402b. The actuating element 1410 of the stent device 1400 is connected to each of the bridge elements 1408a, 1408b that are coupled to the respective apposition ends 1402a, 1402b. The actuating element 1410 is configured to change length responsive to energy changes and/or temperature changes or using mechanical means. The disclosed stent devices include covered stent devices that form a Venturi-type luminal constriction of the vessel wall.

The length of the stent device 1400 is optionally selected to match a physiological size of the target vessel (e.g., length and curves) and/or to ensure good contact with vessel walls. The length of middle portion 1406 may be, for example, 0.5 mm, 1 mm, 2 mm, 3 mm, 5 mm or any smaller, intermediate or larger length, for example selected to achieve desired flow dynamics. The inner diameter of the flared portions 1402a, 1402b can be between 2 mm and 30 mm such as 5 mm, 10 mm, 15 mm, 20 mm or any larger, smaller or intermediate diameter selected to match the vessel diameter. The inner diameter of the middle portion 1406 may be, for example, 1 mm, 2 mm, 3 mm, 5 mm, 10 mm or any smaller, larger or intermediate diameter selected to achieve desired flow dynamics and/or a pressure differential across the stent device 1400. In some implementations, the ratio between the cross-section of narrowed portion 1406 and the flared portions 1402a, 1402b is 0.9, 0.8, 0.6, 0.4, 0.2 or any larger, smaller or intermediate ratio selected to achieve desired flow dynamics and/or a pressure differential across the stent device 1400. While a circular cross-section is shown, other cross-sections may be used, for example, polygona and ellipsoid. A potential advantage of non-circular cross-sections is that the stent device 1400 may be less likely to migrate axially and/or rotate. In some implementations, the apposition ends 1402a, 1402b are asymmetric having different sizes.

The stent device 1400 adjusts the cross-section of the reduced orifice portion 1406 of the stent device 1400 between the apposition ends 1402a, 1402b by changing the length of the actuating element 1410. Shortening the actuating element 1410 causes the apposition ends 1402a, 1402b to approximate which causes the reduced orifice section 1406 to reduce its diameter and circumferential size. Conversely, lengthening the actuating element 1410 increases the size of the passageway through the reduced orifice section 1406 formed by the stent device 1400.

FIGS. 15A, 15B, 15C, and 15D illustrate an example of reducing the size of the passageway through an example stent device 1500. FIGS. 15A and 15C illustrate a top view and side view of the stent device 1500 in a first configuration and FIGS. 15B and 15D illustrate a top view and side view of the stent device 1500 in a second configuration that provides a reduced passageway through the stent device 1500 relative to the first configuration. In FIGS. 15A and 15C, the actuating element 1510 has a first length, L1, which causes the bridge elements 1508a, 1508b to hold the apposition ends 1502a, 1502b away from each other which causes the middle portion 1506 of the stent device 1500 to form a lumen with a diameter, D1. In FIGS. 15B and 15D, the actuating element 1510 is activated (e.g., using heat or electrical energy) causing the actuating element 1510 to contract to a second length, L2 (i.e., L2 is less than L1), shown by the vertical arrows in the figure. Contraction of the actuating element 1510 causes the bridge elements 1508a, 1508b to pull the apposition ends 1502a, 1502b toward each other to cause the middle portion 1506 of the stent device 1500 to contract, shown by the horizontal arrows in the figure, thereby forming a lumen with a diameter, D2 (i.e., D2 is less than D1).

The actuating element 1510 can be made of a shape memory alloy. The actuating element 1510 can be in the martensitic state at body temperature. Once deployed, the actuating element 1510 can be preloaded and stretched to an elongated state. Upon application of heat to the actuating element 1510 (with the temperature above a predefined temperature), the actuating element 1510 is configured to shrink as the material transforms to the austenite state. The shrinking of the actuating element 1510 causes the stent device 1500 to shorten in length. The shortening of the stent device 1500 produces orifice shrinkage. As a result, the reduced cross-sectional area allows less fluid flow through the venous vessel, leading to lower venous return in the systemic circulatory system. This reduction may be advantageous in slowing down the recruitment of venous blood into the stressed blood volume whereas under normal physiological conditions, exercise or other stimulation may increase the blood volume, which can cause pulmonary congestion for certain patients. In some implementations, the A_{f} temperature of the shape memory alloy can be greater than the body temperature (e.g., A_{f}>45° C.) so that the shape memory alloy of the actuating element 1510 is in the martensitic state at body temperature. The stent device 1500 can be covered with any suitable biocompatible material that is biologically compatible with the patient.

The stent device 1500 is configured to control the size of the lumen (e.g., the diameter of the narrow portion 1506) by changing the axial dimension of the stent device 1500. The actuating element 1510 changes size along the axial direction which causes the structure of the stent device 1500 to change. The resulting change causes a change in the size of the orifice or lumen through the stent device 1500. Thus, controlling the size of the axial dimension of the stent device 1500 changes the size of the passageway through the stent device 1500 to control flow through the stent device 1500.

In some implementations, the stent device 1500 is formed from a frame that acts as a spring. In such implementations, releasing or lengthening the actuating element 1510 causes the frame to expand or lengthen back to a biased shape or size, which in turn changes the size of the orifice. In some implementations, the bridge elements 1508a, 1508b can be sutures or any suitable material to provide a mechanical connection between the stent or frame and the actuating element 1510. The stent device 1500 includes anchors 1504a, 1504b to anchor the apposition ends 1502a, 1502b to the walls of the vessel in which it is implanted. Contracting the actuating element 1510 pulls the anchored apposition ends 1502a, 1502b towards one another causing the middle portion 1506 to contract. Contracting the actuating element 1510 thus reduces the size of the orifice.

To actuate the actuating element, various means may be employed. For example, the actuating element 1510 can be mechanically actuated. In such implementations, a torque catheter can interface with the actuating element 1510 (e.g., a screw). Torquing the catheter causes the screw to rotate causing the bridge elements 1508a, 1508b to approximate or to move away from each other, depending on the direction of rotation. As another example, the actuating element 1510 can be electrically actuated. Electrical power can be applied to the actuating element 1510 which can cause the actuating element 1510 to move the bridge elements 1508a, 1508b to cause the middle portion 1506 of the stent to change size. As another example, the actuating element 1510 can be actuated using temperature. The actuating element 1510 can include a shape memory alloy (e.g., a Nitinol wire) that has a transformation temperature above body temperature. Heated saline or electrical power can be used to heat the actuating element 1510 to change the size of the passageway through the stent device.

In some implementations, rather than an hourglass shape, the stent device 1500 can be shaped similar to a bulging cylinder, with a bulge at a middle portion of the stent device. Similar to the hourglass shape, an actuating element oriented axially along the stent can be actuated to lengthen or contract, which moves the apposition ends thereby causing the bulge or middle portion to contract (when the actuating element lengthens) or expand (when the actuating element contracts.

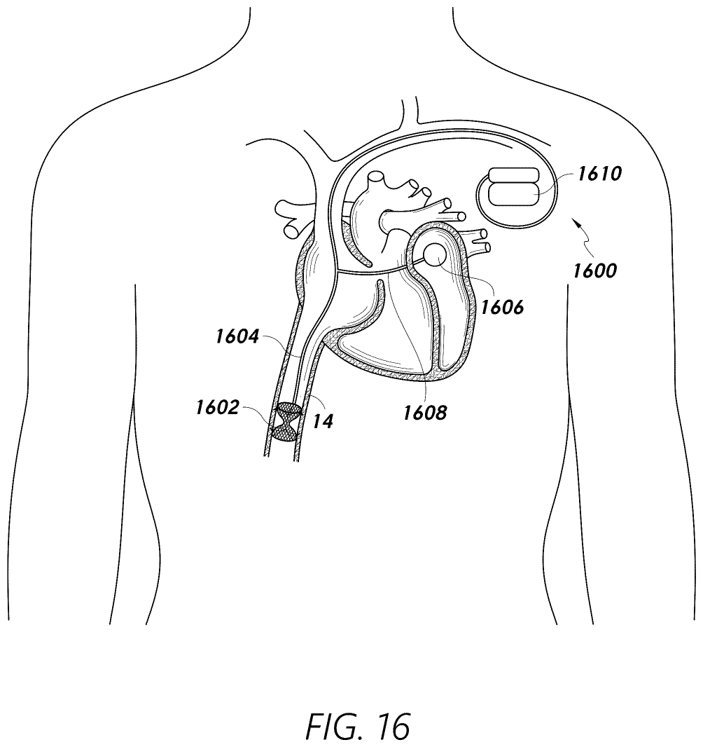

FIG. 16 illustrates an example variable orifice flow control system 1600 that includes a control device 1610 to deliver energy to a stent device 1602 (e.g., the stent device of FIG. 14 or 15), or more accurately, to an actuating element of the stent device 1602. Delivery of energy to the stent device 1602 causes a phase transformation of the actuating element from martensite to austenite. This can lengthen or shorten the actuating element, thereby respectively increasing or decreasing the size of the passageway through the stent device 1602 and, as a result, through the venous vessel 14 in which the stent device 1602 is implanted. This allows the variable orifice flow control system 1600 to control stressed blood volume recruitment. The control device 1610 can operate using feedback from physiological sensors 1606. For example, the control device 1610 can receive physiological signals from one or more sensors 1606 and use these signals as feedback to an algorithm that controls the timing of the modulation of the orifice area of the stent device 1602. The physiological sensors 1606 can be implanted in the heart, such as the right atrium, for example. The stent device 1602 of the variable orifice flow control system 1600 can be implanted in a venous vessel (e.g., the IVC 14) and the control device 1610 can be communicatively coupled to the stent device 1602 and to the physiological sensor 1606 implanted in the left atrium.

The control device 1610 can be coupled to the stent device 1602 wirelessly or through flexible leads 1604. The control device 1610 can include a battery, capacitor, and control circuit. The capacitor is charged by the battery for delivery of energy to the stent device 1602, similar to an implantable cardioverter defibrillator controller. The control circuit provides a signal to discharge the capacitor to the actuating element based on a control algorithm. The control device then recharges the capacitor.

The constriction of the orifice of the stent device 1602 can be programmed to respond to physiological signals. Accordingly, the control device 1610 can include or can be coupled to the one or more physiological sensors 1606 wirelessly or through flexible leads 1608. The physiological sensors 1606 can include a pressure sensor, for example. A pressure sensor can be implanted into the left atrium and can be connected to the control device 1610. In such an implementation, the control device 1610 can deliver energy to the stent device 1602 based at least in part on the pressure signal. Other physiological parameters can also be derived from the pressure sensor 1606, such as heart rate, and can be used to assist the decision making provided by the control algorithm.

Thus, the variable orifice flow control system 1600 includes the stent device 1602 and the control device 1610. The stent device 1602 includes two apposition ends, a variable orifice portion connecting the two apposition ends and situated between the apposition ends to form a central lumen, bridge elements coupled to each apposition end, and an actuating element coupled to the bridge elements of each apposition end. The actuating element is configured to change length and the variable orifice portion is configured to change the size of the cross-sectional area of the central lumen due to the change in length of the actuating element. The variable orifice flow control system 1600 also includes the control device 1610 that is electrically coupled to the actuating element and configured to deliver energy to the actuating element to change the length of the actuating element. The actuating element can be a shape memory alloy. The actuating element can be configured to shorten to constrict the central lumen. The control device 1610 is configured to receive physiological signals and to implement a control algorithm that changes the size of the central lumen in response to the physiological signals. The variable orifice flow control system 1600 is configured to modulate stressed blood volume and/or to reduce recruitment of blood from the splanchnic system. The stent device 1602 can be placed in any vessel where it is advantageous or desirable to control blood flow through the vessel.

In some implementations, the stent device 1602 is formed by cutting out of a sheet of metal or a tube, for example, using laser, water cutting, chemical erosion or metal stamping (e.g., with the result being welded to form a tube). In some implementations, the stent device 1602 is woven (e.g., of metal or plastic fiber), for example, using methods well known in the art. In some implementations, the narrowed portion is made using a different method from the flared portions. For example, the flared portions can be woven and the narrowed portion can be cut from sheet metal. In some implementations, the stent device 1602 can be cut out of a sheet and then spirally twisted around a mandrel to form the shape of the stent device 1602. In some implementations, the stent device 1602 is cut out of a tube, with the flared portions being spiral cuts and the narrowed portion being a ring cut. In some implementations, the stent device 1602 is formed as a coil spring, with axially varying relaxation positions.

The stent device 1602 can be delivered to a targeted destination using a transcatheter approach. Upon attaining its destination, a balloon catheter may be used to encourage the stent device 1602 to attain its contoured shape. As the balloon presses against the lumen of the stent device 1602, the flared portions expand to contact the vessel wall. Various methods for inhibiting undesirable expansion of the narrow portion may be implemented.

The stent device 1602 can include a covering. The covering can be configured to restrict blood flow through the surface of stent device. The covering can be attached to the stent device 1602 at several points (e.g., at the narrow portion and/or the flare portions). In some implementations, the covering is integrated into the structure of the stent device 1602 so that it substantially spans the open areas of the mesh. Examples of materials that can be used in the covering include GORTEX®, latex and/or silicone, on the inside and/or outside of the stent device 1602. Thus, the stent device 1602 forms a conduit by encapsulating the frame in a covering, such as a synthetic biocompatible material. The stent device 1602 is configured to be lodged securely within a vessel or other passage formed between vessels or in the atrial septum to provide variable flow control of blood within the vessel or between vessels.

### Additional Features and Embodiments

As disclosed herein, shape memory metals can include Nitinol or any other suitable material known in the art. Support structures and frames can be constructed using any suitable method including being laser cut from a tube of shape memory metal. As used herein, biocompatible materials may include, for example and without limitation, a sheet of a polymer such as expanded polytetrafluoroethylene (“ePTFE”), polytetrafluoroethylene (“PTFE”,) silicone, polycarbonate urethane, DACRON (polyethylene terephthalate), Ultra High Molecular Weight Polyethylene (UHMWPE), or polyurethane. The biocompatible material may also be a metal, ceramic, carbon nanotube array or any other suitable biocompatible material.

Embodiments of the shunt devices disclosed herein may include anchors with different combinations and configurations of circumferential ring and axial strut elements. These configurations may yield other shunt lumen geometries. In some implementations, the anchors disclosed herein may be made of a self-expanding polymer. In some implementations, the disclosed anchors need not be self-expanding, and may be made from a plastically deformable biocompatible metal such as 316 L stainless steel, cobalt chromium alloys, or any other such suitable materials known to those skilled in the art. Such a deformable shunt anchor may be delivered by an expanding member, such as a balloon, that is configured to achieve the desired luminal geometry. The deformable anchor may be designed to expand prismatically or at certain localized sites where ductile hinges are configured for more selected expansion.

Although certain preferred embodiments and examples are disclosed below, inventive subject matter extends beyond the specifically disclosed embodiments to other alternative embodiments and/or uses, and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

The above description of embodiments of the disclosure is not intended to be exhaustive or to limit the disclosure to the precise form disclosed above. While specific embodiments, and examples, are described above for illustrative purposes, various equivalent modifications are possible within the scope of the disclosure, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel or may be performed at different times.

Certain terms of location are used herein with respect to the various disclosed embodiments. Although certain spatially relative terms, such as “outer,” “inner,” “upper,” “lower,” “below,” “above,” “vertical,” “horizontal,” “top,” “bottom,” and similar terms are used herein to describe a spatial relationship of one device/element or anatomical structure relative to another device/element or anatomical structure, it is understood that these terms are used herein for ease of description to describe the positional relationship between element(s)/structures(s), as illustrated in the drawings. Spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited. In some contexts, description of an operation or event as occurring or being performed “based on,” or “based at least in part on,” a stated event or condition can be interpreted as being triggered by or performed in response to the stated event or condition.

With respect to the various methods and processes disclosed herein, although certain orders of operations or steps are illustrated and/or described, it should be understood that the various steps and operations shown and described may be performed in any suitable or desirable temporal order. Furthermore, any of the illustrated and/or described operations or steps may be omitted from any given method or process, and the illustrated/described methods and processes may include additional operations or steps not explicitly illustrated or described.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above but should be determined only by a fair reading of the claims that follow.

Unless the context clearly requires otherwise, throughout the description and the claims, the terms “comprise,” “comprising,” “have,” “having,” “include,” “including,” and the like are to be construed in an open and inclusive sense, as opposed to a closed, exclusive, or exhaustive sense; that is to say, in the sense of “including, but not limited to.”

The word “coupled”, as generally used herein, refers to two or more elements that may be physically, mechanically, and/or electrically connected or otherwise associated, whether directly or indirectly (e.g., via one or more intermediate elements, components, and/or devices. Additionally, the words “herein,” “above,” “below,” and words of similar import, when used in this application, shall refer to this application as a whole, including any disclosure incorporated by reference, and not to any particular portions of the present disclosure. Where the context permits, words in present disclosure using the singular or plural number may also include the plural or singular number, respectively.

The word “or” in reference to a list of two or more items, that word covers all of the following interpretations of the word: any of the items in the list, all of the items in the list, and any combination of the items in the list. Furthermore, as used herein, the term “and/or” used between elements (e.g., between the last two of a list of elements) means any one or more of the referenced/related elements. For example, the phrase “A, B, and/or C” means “A,” “B,” “C,” “A and B,” “A and C,” “B and C,” or “A, B, and C.”

As may be used herein, the terms “substantially” and “approximately” provides an industry-accepted tolerance for its corresponding term and/or relativity between items. For some industries, an industry-accepted tolerance is less than one percent, while for other industries, the industry-accepted tolerance may be 10 percent or more. Other examples of industry-accepted tolerances range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than approximately +/−1%). Some relativity between items may range from a difference of less than a percentage level to a few percent. Other relativity between items may range from a difference of a few percent to magnitude of differences.

One or more embodiments have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

The one or more embodiments are used herein to illustrate one or more aspects, one or more features, one or more concepts, and/or one or more examples. A physical embodiment of an apparatus, an article of manufacture, a machine, and/or of a process may include one or more of the aspects, features, concepts, examples, etc. described with reference to one or more of the embodiments discussed herein. Further, from figure to figure, the embodiments may incorporate the same or similarly named functions, steps, modules, etc. that may use the same, related, or unrelated reference numbers. The relevant features, elements, functions, operations, modules, etc. may be the same or similar functions or may be unrelated.

### Example Implementations

Example 1: A variable orifice flow control system comprising: an endovascular covered stent device comprising: two apposition ends; a variable orifice portion connecting the two apposition ends and situated between the apposition ends to form a central lumen; bridge elements coupled to each apposition end; and an actuating element coupled to the bridge elements of each apposition end, wherein the actuating element is configured to change length and the variable orifice portion is configured to change a size of a cross-sectional area of the central lumen due to the change in length of the actuating element; and a control system that is electrically coupled to the actuating element and configured to deliver energy to the actuating element to change the length of the actuating element.

Example 2: The variable orifice flow control system of any example herein, in particular example 1, wherein the actuating element is a shape memory alloy.

Example 3: The variable orifice flow control system of any example herein, in particular any of examples 1 to 2, wherein the actuating element is configured to shorten to constrict the central lumen.

Example 4: The variable orifice flow control system of any example herein, in particular any of examples 1 to 3, wherein the control system is configured to receive physiological signals and to implement a control algorithm that changes the size of the central lumen in response to the physiological signals.

Example 5: The variable orifice flow control system of any example herein, in particular example 4, wherein the physiological signals comprise pressure measurements acquired in a left atrium.

Example 6: The variable orifice flow control system of any example herein, in particular any of examples 1-5, wherein the control system is configured to heat the actuating element to constrict the actuating element.

Example 7: The variable orifice flow control system of any example herein, in particular any of examples 1-7, wherein the control system is configured to deliver electrical energy to the actuating element to constrict the actuating element.

Example 8: The variable orifice flow control system of any example herein, in particular any of examples 1-7 further comprising flexible electrical leads that electrically couple the actuating element to the control system.

Example 9: The variable orifice flow control system of any example herein, in particular any of examples 1-8, wherein the actuating element comprises a shape memory alloy configured to be in a martensitic state at body temperature.

Example 10: The variable orifice flow control system of any example herein, in particular any of examples 1-9, wherein the endovascular covered stent device is configured to be implanted in the inferior vena cava of a patient.

Example 11: A shunt device configured to alleviate pressure overload of a heart organ, the shunt device comprising: an inlet port; an outlet port; two or more leaflets to form the outlet port, the two or more leaflets pre-formed to hold a closed state at or under a threshold pressure differential, the two or more leaflets configured to open to allow blood flow through the shunt device in response to a pressure that exceeds the threshold pressure differential; and two or more commissure posts to couple the two or more leaflets to the shunt device, the two or more commissure posts extending from the inlet port to the outlet port.

Example 12: The shunt device of any example herein, in particular example 11, wherein the shunt device comprises metallic alloys.

Example 13: The shunt device of any example herein, in particular any of examples 11-12, wherein the shunt device comprises plastics.

Example 14: The shunt device of any example herein, in particular any of examples 11-13, wherein the shunt device is configured for placement at a junction between the superior vena cava and pulmonary artery.

Example 15: The shunt device of any example herein, in particular any of examples 11-14, wherein the shunt device is configured to alleviate right ventricle pressure.

Example 16: The shunt device of any example herein, in particular any of examples 11-15, wherein the two or more leaflets are configured to remain closed when a pressure differential between the inlet port and the outlet ports is at or below 20 mmHg.

Example 17: The shunt device of any example herein, in particular example 16, wherein the two or more leaflets are configured to open responsive to the pressure differential exceeding 20 mmHg.

Example 18: The shunt device of any example herein, in particular any of examples 11-17 further comprising a structural support frame, the two or more commissure ports extending from the structural support frame, the two or more leaflets coupled to the structural support frame.

Example 19: The shunt device of any example herein, in particular example 18, wherein the structural support frame includes a flexible portion configured to move with the two or more leaflets.

Example 20: The shunt device of any example herein, in particular any of examples 11-19, wherein the two or more leaflets comprise a biocompatible polymer.

Example 21: A shunt device configured to alleviate pressure overload of a heart organ, the shunt device comprising: an inlet port; an outlet port; a check valve formed from at least three leaflets that form the outlet port, the at least three leaflets pre-formed to hold a closed state at or under a threshold pressure differential, the at least three leaflets configured to open to allow blood flow through the shunt device in response to a pressure that exceeds the threshold pressure differential; at least three commissure posts to couple the at least three leaflets to the shunt device, the at least three commissure posts extending from the inlet port to the outlet port; and at least three cusp posts that regulate opening of the at least three leaflets.

Example 22: The shunt device of any example herein, in particular example 21, wherein the shunt device comprises metallic alloys.

Example 23: The shunt device of any example herein, in particular any of examples 21-22, wherein the shunt device comprises plastics.

Example 24: The shunt device of any example herein, in particular any of examples 21-23, wherein the shunt device is configured for placement at a junction between the superior vena cava and pulmonary artery, at an interatrial septum of the heart organ, or between a left atrium and coronary sinus of the heart organ.

Example 25: The shunt device of any example herein, in particular any of examples 21-24, wherein the check valve comprises a variable flow control device that provides a plurality of flow states.

Example 26: The shunt device of any example herein, in particular any of examples 21-25, wherein the shunt device is configured to alleviate right ventricle pressure.

Example 27: The shunt device of any example herein, in particular any of examples 21-26, wherein the at least three leaflets are configured to remain closed when a pressure differential between the inlet port and the outlet ports is at or below 20 mmHg.

Example 28: The shunt device any example herein, in particular example 27, wherein the at least three leaflets are configured to open responsive to the pressure differential exceeding 20 mmHg.

Example 29: The shunt device of any example herein, in particular any of examples 21-28 further comprising a structural support frame that forms a tri-leaflet variable orifice support with three stiff posts corresponding to the at least three commissure ports and three flexible posts corresponding to the at least three cusp posts.

Example 30: The shunt device of any example herein, in particular example 29, wherein the check valve forms a shamrock shape when the check valve is open.

Example 31: A shunt device configured to provide a variable orifice, the shunt device comprising: a shunt frame that forms an inlet port and an outlet port and that is configured to support a shunt opening; a leaflet support structure coupled to the support frame; a leaflet comprising a soft tissue or polymer membrane, the leaflet coupled to the leaflet support structure, the leaflet support structure configured to deflect in response to a pressure differential or a pressure gradient imposed on the leaflet; and a wrapping membrane configured to wrap the support frame to regulate a flow rate through the shunt device, wherein the leaflet and the leaflet support structure cooperate to form a flapper check valve that provides a variable orifice flow responsive to the pressure gradient imposed on the leaflet.

Example 32: The shunt device of any example herein, in particular example 31, wherein the leaflet support structure comprises an elastic material or a super-elastic alloy configured so that a cracking pressure of the shunt device can be set to a targeted value.

Example 33: The shunt device of any example herein, in particular example 32, wherein the cracking pressure is greater than or equal to 1 mmHg.

Example 34: The shunt device of any example herein, in particular any of examples 32-33, wherein the cracking pressure is less than or equal to 20 mmHg.

Example 35: The shunt device of any example herein, in particular any of examples 31-34, wherein the leaflet support structure is made of a biocompatible metal alloy or engineering plastics that is configured to deflect in response to the pressure gradient imposed on the leaflet attached to the leaflet support structure.

Example 36: The shunt device of any example herein, in particular any of examples 31-35, wherein the leaflet support structure is configured to partially open responsive to the pressure gradient imposed on the leaflet exceeding a first threshold pressure gradient.

Example 37: The shunt device of any example herein, in particular example 36, wherein the leaflet support structure is configured to fully open responsive to the pressure gradient imposed on the leaflet exceeding a second threshold pressure gradient that is greater than the first threshold pressure gradient.

Example 38: The shunt device of any example herein, in particular example 37, wherein the leaflet support structure is configured to close responsive to the pressure gradient imposed on the leaflet being less than or equal to the first threshold pressure gradient.

Example 39: The shunt device of any example herein, in particular example 38, wherein a position of the leaflet support structure between a closed position and a fully open position is related to the pressure gradient imposed on the leaflet, the pressure gradient greater than the first threshold pressure gradient and less than the second threshold pressure gradient.

Example 40: The shunt device of any example herein, in particular any of examples 31-39, wherein the shunt device is configured for placement between pulmonary circulation vessels and venous vessels.

Example 41: A method for deploying and adjusting a shunt device with an adjustable orifice, the method comprising: implanting a shunt device in a targeted location in a patient, the shunt device implanted in a crimped configuration, the shunt device including a structural support frame forming an orifice; expanding the shunt device so that the orifice has a first diameter; monitoring patient hemodynamics; and adjusting a diameter of the shunt device to achieve a target flow rate through the orifice, the adjusted diameter being a second diameter different from the first diameter.

Example 42: The method of any example herein, in particular example 41, further comprising adjusting the diameter of the shunt device to a third diameter different from the first diameter and the second diameter responsive to measurements of patient hemodynamics indicating that the flow rate through the orifice is different from a targeted flow rate.

Example 43: The method of any example herein, in particular example 42, wherein expanding the shunt device comprises using a balloon catheter to mechanically expand the shunt device and adjusting the diameter of the shunt device comprises using the balloon catheter to deliver heat to the shunt device to cause a shape memory alloy to change shape.

Example 44: The method of any example herein, in particular example 43, wherein adjusting to the third diameter comprises using the balloon catheter that delivered the heat to the shunt device to mechanically expand the shunt device.

Example 45: The method of any example herein, in particular any of examples 41-44, wherein the shunt device comprises a homogeneous shape memory alloy that is configured to operate in its martensitic phase at body temperature.

Example 46: The method of any example herein, in particular any of examples 41-45, wherein the shunt device comprises a structural support frame having a nominally uniform chemical composition that is selectively heat treated at a plurality of locations to alter local properties between martensitic and austenitic phases at body temperature.

Example 47: The method of any example herein, in particular any of examples 41-46, wherein the shunt device comprises a locally variable chemical stoichiometry such that portions of the shunt device operate in a martensitic phase at body temperature and different portions of the shunt device operate in an austenitic phase at body temperature.

Example 48: The method of any example herein, in particular example 47, wherein the locally variable chemical stoichiometry is achieved by selectively depositing material on portions of the shunt device.

Example 49: The method of any example herein, in particular any of examples 41-48, wherein the shunt device further comprises: a balloon-expandable structural support frame comprising metallic alloys; and a super-elastic anchor that provides fixation of the shunt device into the targeted location.

Example 50: The method of any example herein, in particular example 49, wherein the balloon-expandable structural support frame is integrated with the super-elastic anchor.

What is claimed is:

1. A variable orifice flow control system comprising:
an endovascular covered stent device comprising:
two apposition ends;
a variable orifice portion connecting the two apposition ends and situated between the apposition ends to form a central lumen;
bridge elements coupled to each apposition end; and
an actuating element coupled to the bridge elements of each apposition end,
wherein the actuating element is configured to change length and the variable orifice portion is configured to change a size of a cross-sectional area of the central lumen due to the change in length of the actuating element; and
a control system that is electrically coupled to the actuating element and configured to deliver energy to the actuating element to change the length of the actuating element.

2. The variable orifice flow control system of claim 1, wherein the actuating element is a shape memory alloy.

3. The variable orifice flow control system of claim 1, wherein the actuating element is configured to shorten to constrict the central lumen.

4. The variable orifice control system of claim 1, wherein the control system is configured to receive physiological signals and to implement a control algorithm that changes the size of the central lumen in response to the physiological signals.

5. The variable orifice control system of claim 4, wherein the physiological signals comprise pressure measurements acquired in a left atrium.

6. A shunt device configured to alleviate pressure overload of a heart organ, the shunt device comprising:
an inlet port;
an outlet port;
two or more leaflets to form the outlet port, the two or more leaflets pre-formed to hold a closed state at or under a threshold pressure differential, the two or more leaflets configured to open to allow blood flow through the shunt device in response to a pressure that exceeds the threshold pressure differential; and
two or more commissure posts to couple the two or more leaflets to the shunt device, the two or more commissure posts extending from the inlet port to the outlet port.

7. The shunt device of claim 6, wherein the shunt device comprises metallic alloys.

8. The shunt device of claim 6, wherein the shunt device comprises plastics.

9. The shunt device of claim 6, wherein the shunt device is configured for placement at a junction between the superior vena cava and pulmonary artery.

10. The shunt device of claim 6, wherein the shunt device is configured to alleviate right ventricle pressure.

11. The shunt device of claim 6, wherein the two or more leaflets are configured to remain closed when a pressure differential between the inlet port and the outlet ports is at or below 20 mmHg.

12. The shunt device of claim 11, wherein the two or more leaflets are configured to open responsive to the pressure differential exceeding 20 mmHg.

13. The shunt device of claim 6 further comprising a structural support frame, the two or more commissure ports extending from the structural support frame, the two or more leaflets coupled to the structural support frame.

14. The shunt device of claim 13, wherein the structural support frame includes a flexible portion configured to move with the two or more leaflets.

15. The shunt device of claim 6, wherein the two or more leaflets comprise a biocompatible polymer.

16. A flow reducing implant having a variable orifice, comprising:
a self-expanding tubular structure having first and second end portions and a variable orifice between the first and second end portions, the first and second end portions having a diameter sized for engaging an inner wall of a blood vessel, the variable orifice having a diameter less than the first and second end portions and defining the flow reducing region;
a cover disposed along a surface of the tubular structure; and
an actuating element for adjusting the diameter of the variable orifice.

17. The flow reducing implant of claim 16, wherein adjusting a length of the actuating element affects the diameter of the variable orifice.

18. The flow reducing implant of claim 17, further comprising a control system electrically coupled to the actuating element for changing the length of the actuating element.

19. The flow reducing implant of claim 18, further comprising barbs shaped for anchoring the tubular structure to the inner wall of the blood vessel.

20. The flow reducing implant of claim 18, wherein the tubular structure is sized for implantation in a vessel of the splanchnic system for reducing blood flow from the splanchnic system, thereby alleviating symptoms of pulmonary congestion.
